(19) United States
(12) Patent Application Publication
(10) Pub. No.: US 2006/0275792 A1
(43) Pub. Date: Dec. 7, 2006

(54) Enhancement of nucleic acid amplification using double-stranded DNA binding proteins

(76) Inventors: Jun E. Lee, San Diego, CA (US); Robert J. Potter, San Marcos, CA (US); Sandrine M. M. Javorschi-Miller, La Jolla, CA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/365,383

(22) Filed: Feb. 28, 2006

Related U.S. Application Data
(63) Continuation-in-part of application No. 10/987,453, filed on Nov. 15, 2004.

(51) Int. Cl. C12Q 1/68; C12P 19/34

(52) U.S. Cl. 435/6; 435/91.2

(57) ABSTRACT

Disclosed are methods and materials for enhancing the efficiency, specificity and/or specificity of nucleic acid amplification reactions (e.g., PCR amplification reactions). The disclosed inventions involve the use of nonspecific double-stranded DNA binding proteins in amplification reactions. The methods described herein are useful in a wide variety of molecular biology applications including allele specific PCR.

### RELATED APPLICATIONS

This application claims priority to U.S. patent application Ser. No. 10/987,453, filed Nov. 15, 2004, and to U.S. Provisional Application Ser. No. 60/015,720, entitled “Enhancement of nucleic acid amplification using double-stranded DNA binding proteins”, filed Feb. 28, 2005. The entire contents of both of these priority applications is incorporated herein by reference.

### FIELD OF THE INVENTION

The present invention relates to the field of molecular biology. In particular, the present invention relates to the use of double-stranded DNA binding proteins for improving the efficiency, sensitivity and/or specificity of nucleic acid amplification (e.g., PCR) reactions.

### BACKGROUND

The amplification of nucleic acid molecules generally involves the extension of a nucleic acid target molecule using an oligonucleotide primer in a reaction catalyzed by polymerase. For example, Panet and Khorana (J. Biol. Chem. 249:5213-5221 (1974)) demonstrate the replication of deoxyribopolynucleotide templates bound to cellulose. Kieppe et al., (J. Mol. Biol. 56:341-361 (1971)) disclose the use of double- and single-stranded DNA molecules as templates for the synthesis of complementary DNA.

Other known nucleic acid amplification procedures include transcription based amplification systems (Kwoh, D. et al., Proc. Natl. Acad. Sci. USA 86:1173 (1989); PCT appl. WO 88/10315). Schemes based on ligation (“Ligation Chain Reaction” (“LCR”)) of two or more oligonucleotides in the presence of a target nucleic acid having a sequence complementary to the sequence of the product of the ligation reaction have also been used (Wu, D. Y. et al., Genomics 4:560 (1989)). Other suitable methods for amplifying nucleic acid based on ligation of two oligonucleotides after annealing to complementary nucleic acids are known in the art.

PCT appl. WO 89/06700 discloses a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts.

EP 329,822 discloses an alternative amplification procedure termed Nucleic Acid Sequence-Based Amplification (NASBA). NASBA is a nucleic acid amplification process comprising cyclically synthesizing single-stranded RNA (“ssRNA”), ssDNA, and double-stranded DNA (dsDNA). The ssRNA is a first template for a first primer oligonucleotide, which is elongated by reverse transcriptase (RNA dependent DNA polymerase). The RNA is then removed from the resulting DNA:RNA duplex by the action of ribonuclease H (RNase H, an RNase specific for RNA in a duplex with either DNA or RNA). The resultant ssDNA is a second template for a second primer. The second primer includes the sequences of an RNA polymerase promoter (exemplified by T7 RNA polymerase) located 5′ to the primer sequence which hybridizes to the ssDNA template. This primer is then extended by a DNA polymerase (exemplified by the large “Klenow” fragment of E. coli DNA polymerase I), resulting in the production of a dsDNA molecule, having a sequence identical to that of the portion of the original RNA located between the primers and having, additionally, at one end, a promoter sequence. This promoter sequence can be used by the appropriate RNA polymerase to make many RNA copies of the DNA. These copies can then re-enter the cycle leading to very swift amplification. With the proper choice of enzymes, this amplification can be done isothermally without the addition of enzymes at each cycle. Because of the cyclical nature of this process, the starting sequence can be chosen to be in the form of either DNA or RNA.

U.S. Pat. No. 5,455,166 and EP 684 315 disclose a method called Strand Displacement Amplification (SDA). This method is performed at a single temperature and uses a combination of a polymerase, an endonuclease and a modified nucleoside triphosphate to amplify single-stranded fragments of the target DNA sequence. A target sequence is fragmented, made single-stranded and hybridized to a primer that contains a recognition site for an endonuclease. The primer:target complex is then extended with a polymerase enzyme using a mixture of nucleoside triphosphates, one of which is modified. The result is a duplex molecule containing the original target sequence and an endonuclease recognition sequence. One of the strands making up the recognition sequence is derived from the primer and the other is a result of the extension reaction. Since the extension reaction is performed using a modified nucleotide, one strand of the recognition site is modified and resistant to endonuclease digestion. The resultant duplex molecule is then contacted with an endonuclease which cleaves the unmodified strand causing a nick. The nicked strand is extended by a polymerase enzyme lacking 5′-3′ exonuclease activity resulting in the displacement of the nicked strand and the production of a new duplex molecule. The new duplex molecule can then go through multiple rounds of nicking and extending to produce multiple copies of the target sequence.

The most widely used method of nucleic acid amplification is the polymerase chain reaction (PCR). A detailed description of PCR is provided in the following references: Mullis, K. et al., Cold Spring Harbor Symp. Quant. Biol. 51:263-273 (1986); EP 50,424; EP 84,796; EP 258,017; EP 237,362; EP 201,184; U.S. Pat. No. 4,683,202; U.S. Pat. No. 4,582,788; and U.S. Pat. No. 4,683,194. In its simplest form, PCR involves the amplification of a target double-stranded nucleic acid sequence. The double-stranded sequence is denatured and an oligonucleotide primer is annealed to each of the resultant single strands. The sequences of the primers are selected so that they will hybridize in positions flanking the portion of the double-stranded nucleic acid sequence to be amplified. The oligonucleotides are extended in a reaction with a polymerase enzyme, nucleotide triphosphates and the appropriate cofactors resulting in the formation of two double-stranded molecules each containing the target sequence. Each subsequent round of denaturation, annealing and extension reactions results in a doubling of the number of copies of the target sequence as extension products from earlier rounds serve as templates for subsequent replication steps. Thus, PCR provides a method for selectively increasing the concentration of a nucleic acid molecule having a particular sequence even when that molecule has not been previously purified and is present only in a single copy in a particular sample. The method can be used to amplify either single- or double-stranded nucleic acids. The essence of the method involves the use of two oligonucleotides to serve as primers for the template dependent, polymerase-mediated replication of the desired nucleic acid molecule.

PCR has found numerous applications in the fields of research and diagnostics. One area in which PCR has proven useful is the detection of single nucleotide mutations by allele specific PCR (ASPCR) (see, for example, U.S. Pat. Nos. 5,639,611 and 5,595,890). As originally described by Wu, et al. (Proceedings of the National Academy of Sciences, USA, 86:2757-2760 (1989)), ASPCR involves the detection of a single nucleotide variation at a specific location in a nucleic acid molecule by comparing the amplification of the target using a primer sequence whose 3′-termini nucleotide is complementary to a suspected variant nucleotide to the amplification of the target using a primer in which the 3′-termini nucleotide is complementary to the normal nucleotide. In the case where the variant nucleotide is present in the target, amplification occurs more efficiently with the primer containing the 3′-nucleotide complementary to the variant nucleotide while in the case where the normal nucleotide is present in the target, amplification is more efficient with the primer containing 3′-nucleotide complementary to the normal nucleotide.

While some amplification-based methods for identifying and quantifying nucleic acids involve a separation step, several allow detection of nucleic acids without separating the labeled primer or probe from the reaction. These methods have numerous advantages compared to gel-based methods, such as gel electrophoresis, and dot-blot analysis, for example, and require less time, permit high throughput, prevent carryover contamination and permit quantification through real time detection. Most of these current methods are solution-based fluorescence methods that utilize two chromophores. These methods utilize the phenomena of fluorescence resonance energy transfer (FRET) in which the energy from an excited fluorescent moiety is transferred to an acceptor molecule when the two molecules are in close proximity to each other. This transfer prevents the excited fluorescent moiety from releasing the energy in the form of a photon of light thus quenching the fluorescence of the fluorescent moiety. When the acceptor molecule is not sufficiently close, the transfer does not occur and the excited fluorescent moiety may then fluoresce. The major disadvantages of systems based on FRET are the cost of requiring the presence of two modified nucleotides in a detection oligonucleotide and the possibility that the efficiency of the quenching may not be sufficient to provide a usable difference in signal under a given set of assay conditions. Other known methods which permit detection without separation are: luminescence resonance energy transfer (LRET) where energy transfer occurs between sensitized lanthanide metals and acceptor dyes (Selvin, P. R., and Hearst, J. D., Proc. Natl. Acad. Sci. USA 91:10024-10028 (1994)); and color change from excimer-forming dyes where two adjacent pyrenes can form an excimer (fluorescent dimer) in the presence of the complementary target, resulting in a detectably shifted fluorescence peak (Paris, P. L. et al., Nucleic Acids Research 26:3789-3793 (1998)).

One method for detecting amplification products without prior separation of product is described in U.S. Pat. No. 5,348,853 and Wang et al., Anal. Chem. 67:1197-1203 (1995), uses an energy transfer system in which energy transfer occurs between two fluorophores on the probe. In this method, detection of the amplified molecule takes place in the amplification reaction vessel, without the need for a separation step. The Wang et al. method uses an “energy-sink” oligonucleotide complementary to the reverse primer. The “energy-sink” and reverse primer oligonucleotides have donor and acceptor labels, respectively. Prior to amplification, the labeled oligonucleotides form a primer duplex in which energy transfer occurs freely. Then, asymmetric PCR is carried out to its late-log phase before one of the target strands is significantly overproduced.

A second method for detection of an amplification product without prior separation of product is the 5′ nuclease PCR assay (also referred to as the TAQMAN® assay) (Holland et al., Proc. Natl. Acad. Sci. USA 88:7276-7280 (1991); Lee et al., Nucleic Acids Res. 21:3761-3766 (1993)). This assay detects the accumulation of a specific PCR product by hybridization and cleavage of a doubly labeled fluorogenic probe (the “TAQMAN®” probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if it hybridizes to the segment being amplified. Cleavage of the probe generates an increase in the fluorescence intensity of the reporter dye. In the TAQMAN® assay, the donor and quencher are preferably located on the 3′- and 5′-ends of the probe, because the requirement that 5′-3′ hydrolysis be performed between the fluorophore and quencher may be met only when these two moieties are not too close to each other (Lyamichev et al., Science 260:778-783 (1993)).

Another method of detecting amplification products (namely MOLECULAR BEACONS) relies on the use of energy transfer using a “beacon probe” described by Tyagi and Kramer (Nature Biotech. 14:303-309 (1996)). This method employs oligonucleotide hybridization probes that can form hairpin structures. On one end of the hybridization probe (either the 5′- or 3′-end), there is a donor fluorophore, and on the other end, an acceptor moiety. In the case of the Tyagi and Kramer method, the acceptor moiety is a quencher, that is, the acceptor absorbs energy released by the donor, but then does not itself fluoresce. Thus, when the beacon is in the open conformation, the fluorescence of the donor fluorophore is detectable, whereas when the beacon is in hairpin (closed) conformation, the fluorescence of the donor fluorophore is quenched. When employed in PCR, the beacon probe, which hybridizes to one of the strands of the PCR product, is in “open conformation,” and fluorescence is detected, while those that remain unhybridized will not fluoresce. As a result, the amount of fluorescence will increase as the amount of PCR product increases, and thus may be used as a measure of the progress of the PCR.

Another method of detecting amplification products which relies on the use of energy transfer is the SUNRISE PRIMER method of Nazarenko et al. (Nucleic Acids Research 25:2516-2521 (1997); U.S. Pat. No. 5,866,336). SUNRISE PRIMERS are based on FRET and other mechanisms of non-fluorescent quenching. SUNRISE PRIMERS consist of a single-stranded primer with a hairpin structure at its 5′-end. The hairpin stem is labeled with a donor/quencher pair. The signal is generated upon the unfolding and replication of the hairpin sequence by polymerase.

These and other amplification based nucleic acid detection methods can benefit from materials and methods designed to make them more sensitive and more discriminating for detecting target nucleic acids.

### SUMMARY OF THE INVENTION

The present invention is based, at least in part, on the surprising finding that non-specific double-stranded DNA (dsDNA) binding proteins can enhance the efficiency, specificity, and/or sensitivity of nucleic acid amplification (e.g., PCR) reactions.

Disclosed are methods for amplifying nucleic acid molecules, comprising contacting the nucleic acid molecule with a thermostable nucleic acid polymerase and a nonspecific double-stranded DNA binding protein, whereby the nucleic acid molecule is amplified. The nucleic acid may be DNA or RNA. In one aspect of this embodiment, the polymerase is selected from the group consisting of Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT®) DNA polymerase, Pyrococcus furiosus (Pfu) DNA polymerase, DEEPVENT® DNA polymerase, Pyrococcus woosii (Pwo) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME®) DNA polymerase, Methanobacterium thermoautotrophicum (Mth) DNA polymerase, mycobacterium DNA polymerase (Mtb, Mlep), Thermococcus zilligii (Tzi) polymerase, and mutants, variants and derivatives thereof. The amplification may be PCR amplification. In one embodiment, the PCR amplification is selected from the group consisting of end-point PCR, qPCR, allele specific amplification, linear PCR, RT-PCR, mutagenic PCR and multiplex PCR. In another embodiment, the nonspecific double-stranded DNA binding protein is SsO7d.

Also disclosed are compositions comprising a nucleic acid molecule, a thermostable nucleic acid polymerase and a nonspecific double-stranded DNA binding protein. The nucleic acid may be DNA or RNA. In one aspect of this embodiment, the polymerase is selected from the group consisting of Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT®) DNA polymerase, Pyrococcus furiosus (Pfu) DNA polymerase, DEEPVENT® DNA polymerase, Pyrococcus woosii (Pwo) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME®) DNA polymerase, Methanobacterium thermoautotrophicum (Mth) DNA polymerase, mycobacterium DNA polymerase (Mtb, Mlep), Thermococcus zilligii (Tzi) polymerase, and mutants, variants and derivatives thereof. In one aspect, the nonspecific double-stranded DNA binding protein is SsO7d.

### BRIEF DESCRIPTION OF THE DRAWINGS

FIG. 1 is a diagram showing single nucleotide polymorphism (SNP) detection of a T/C genotype using universal primers, each primer having a different internal fluorescent label (5-carboxyfluorescein (FAM) or 2′7′-dimethoxy-4′5′-dichloro-6-carboxyfluorescein (JOE). Components include two universal primers, two allele-specific primers and a corresponding common primer which hybridizes to the target nucleic acid. (1) Allele-specific primers anneal to the target nucleic acid and are extended by DNA polymerase. Each primer has a unique 5′ extension identical to one of the fluorescently labeled universal primers. (2) The common primer anneals and is extended, generating the complementary sequences of the 5′ extensions. (3) The universal primers unfold and anneal to the products of the reverse reaction and are extended resulting in allele specific fluorescent signals.

FIGS. 2 show real-time amplification genotyping results of various SNP types using the assay described in FIG. 1. FIGS. 2A-B show genotyping analyis of a G/C polymorphism in the human BAT1 gene; FIGS. 2C-D show genotyping analysis of an A/G polymorphism in the human BAT 3 gene; and FIGS. 2E-F show genotyping analysis of a T/G polymorphism in the human CCNB1(P7) gene. FIGS. 2G, 2H and 2I show allelic discrimination plots of the data presented in FIGS. 2A-B, FIGS. 2C-D and FIGS. 2E-F, respectively.

FIGS. 3A-H show real-time and allelic discrimination analysis of a G/C polymorphism in the human BAT 1 gene using the assay described in FIG. 1 in the absence (FIGS. 3A-C) or presence (FIGS. 3 D-F) of the nonspecific double-stranded DNA binding protein SsO7d from Sulfolobus solfataricus. Comparison of results obtained in a real-time analysis in the presence or absence of SsO7d is shown in FIGS. 3G-H. Lines with an asterisk indicate the presence of SsO7d.

FIGS. 4A-C show raw fluorescence data from the FAM channel for the BAT1 (FIG. 4A), BAT3 (FIG. 4B) and CCNB1 (FIG. 4C) SNPs. Each plot shows the reduction of the background fluorescence with the addition of SsO7d.

FIGS. 5A-D show improvement of the efficiency and specificity of PCR. FIGS. 5A and 5C show a typical amplification plot and standard curve, respectively, with the nonspecific double-stranded binding protein SsO7d. FIGS. 5B and 5D show a typical amplification plot and standard curve, respectively, with SsO7d. PCR efficiency increased from 96.0% (−3.422) without SsO7d (FIG. 5B) to 97.6% (−3.381) with SsO7d (FIG. 5D), and an earlier y-intercept (increased sensitivity) from 38.12 without SsO7d (FIG. 5B) to 38.01 with SsO7d (FIG. 5D). Lastly, signal in the NTCs (no template controls) was detected at a later Ct in the presence of SsO7d, from 37.98±0.01 without SsO7d (FIG. 5A) to 39.76±1.66 with SsO7d.

FIG. 6 is a graph showing that the formation of nonspecific primer dimers in PCR was delayed by SsO7d in a dose-dependent manner.

FIG. 7 is a graph showing that PCR amplification with the combination of Taq polymerase and PA polymerase occurred only in the presence of SsO7d.

### DETAILED DESCRIPTION OF THE INVENTION

The present invention provides methods and materials that involve the use of non-specific dsDNA binding proteins to enhance the efficiency, specificity, and/or sensitivity of nucleic acid amplification (e.g., PCR) reactions.

The following terms shall have the indicated meanings:

Amplification. As used herein, “amplification” refers to any in vitro method for increasing the number of copies (e.g. at least 3-fold) of a nucleotide sequence with the use of a polymerase. Nucleic acid amplification results in the incorporation of nucleotides into a nucleic acid molecule (e.g., DNA) or primer thereby forming a new nucleic acid molecule complementary to the nucleic acid template. The formed nucleic acid molecule and its template can be used as templates to synthesize additional nucleic acid molecules. As used herein, one amplification reaction may consist of many rounds of nucleic acid synthesis. Amplification reactions include those described in the Background section of this application, including, for example, polymerase chain reactions (PCR). One PCR reaction may consist of 5 to 100 “cycles” of denaturation and synthesis of a nucleic acid molecule.

Specificity enhancing group. As used herein “specificity enhancing group” refers to any molecule or group of molecules that causes an oligonucleotide of the present invention to be less and preferably substantially less extendable when the 3′-most nucleotide of the oligonucleotide is substantially not base paired with a nucleotide on the nucleic acid target/template molecule. Any type of group may be used. Preferred examples include, but are not limited to, fluorescent groups, modified nucleotides, nucleotide analogues, small molecules, haptens and the like. Specificity enhancing groups may be attached at any position of the oligonucleotide or be a part of the oligonucleotide at any position (for example, when the specificity enhancing group is a modified nucleotide or nucleotide analogue) so long as they make the oligonucleotide less and preferably substantially less extendable when the 3′-termini nucleotide of the oligonucleotide is substantially not base paired with the corresponding nucleotide of the target/template nucleic acid. Such groups are preferably attached to the primer or oligonucleotide or part of the primer or oligonucleotide at or near the 3′-end of the primer or oligonucleotide, but may be attached or placed at other positions as well. Preferably, they are attached to or located to one or more of the 25 bases adjacent to the 3′-end of the primer or oligonucleotide. In some preferred embodiments, such groups may be attached to or located to one or more of the 20 bases adjacent to the 3′-end of the oligonucleotide, or to or part of the 15 bases adjacent to the 3′-end or to or part of the 10 bases adjacent to the 3′-end or, most preferably to or part of one or more of the five bases adjacent to the 3′-end of the oligonucleotide. In addition, specificity enhancing groups may be attached to or a part of the 3′-most nucleotide so long as the presence of the group does not prevent or inhibit the extension of the primer when the 3′-most nucleotide of the primer is complementary to the corresponding nucleotide on the target/template molecule more than the extension is inhibited when the 3′-most nucleotide is substantially not base paired to the target/template. Any group that can decrease the stability of the duplex (double-stranded complex) formed by the primer and template when the 3′-most nucleotide of the primer or oligonucleotide is not complementary to the corresponding nucleotide of the target/template and/or any group that can make a polymerase less efficient at extending the 3′-end of the oligonucleotide when the 3′-most nucleotide is not complementary to the corresponding nucleotide of the template/target may be used to practice the present invention. In some embodiments, the specificity enhancing groups of the invention may be modified nucleotides or nucleotide analogues incorporated into the sequence of the primer or oligonucleotide. Such modifications may be made at the base, sugar or phosphate portion of the nucleotide and include, but are not limited to, phosphothioate nucleotides, phosphonate nucleotides, peptide nucleic acids and the like. A specificity enhancing group is used, for example, in allele-specific PCR to enhance discrimination.

Polymerase. As used herein “polymerase” refers to any enzyme having a nucleotide polymerizing activity. Polymerases (including DNA polymerases and RNA polymerases) useful in accordance with the present invention include, but are not limited to, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT®) DNA polymerase, Pyrococcus furiosus (Pfu) DNA polymerase, DEEPVENT® DNA polymerase, Pyrococcus woosii (Pwo) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME®) DNA polymerase, Methanobacterium thermoautotrophicum (Mth) DNA polymerase, mycobacterium DNA polymerase (Mtb, Mlep), Thermococcus zilligii (Tzi) polymerase, and mutants, variants and derivatives thereof. The isolation and characterization of Tzi DNA polymerase is described in copending U.S. patent application Ser. No. 11/044,620, filed Jan. 28, 2005, the content of which are incorporated by reference in their entirety. RNA polymerases such as T3, T5 and SP6 and mutants, variants and derivatives thereof may also be used in accordance with the invention. Generally, any type I DNA polymerase may be used in accordance with the invention although other DNA polymerases may be used including, but not limited to, type III or family A, B, C, etc. DNA polymerases.

Polymerases used in accordance with the invention may be any enzyme that can synthesize a nucleic acid molecule from a nucleic acid template, typically in the 5′ to 3′ direction. “3′ to 5′ exonuclease activity” is an enzymatic activity well known to the art. This activity is often associated with DNA polymerases, and is thought to be involved in a DNA replication “editing” or correction mechanism. A “polymerase substantially reduced in 3′ to 5′ exonuclease activity” is defined herein as either (1) a mutated or modified polymerase that has about or less than 10%, or preferably about or less than 1%, of the 3′ to 5′ exonuclease activity of the corresponding unmutated, wild-type enzyme, or (2) a polymerase having a 3′ to 5′ exonuclease specific activity which is less than about 1 unit/mg protein, or preferably about or less than 0.1 units/mg protein. A unit of activity of 3′ to 5′ exonuclease is defined as the amount of activity that solubilizes 10 nmoles of substrate ends in 60 min. at 37° C., assayed as described in the “BRL 1989 Catalogue & Reference Guide,” page 5, with HhaI fragments of lambda DNA 3′-end labeled with [³H]dTTP by terminal deoxynucleotidyl transferase (TdT). Protein is measured by the method of Bradford, Anal. Biochem. 72:248 (1976). As a means of comparison, natural, wild-type T5-DNA polymerase (DNAP) or T5-DNAP encoded by pTTQ19-T5-2 has a specific activity of about 10 units/mg protein while the DNA polymerase encoded by pTTQ19-T5-2(Exo-) (U.S. Pat. No. 5,270,179) has a specific activity of about 0.0001 units/mg protein, or 0.001% of the specific activity of the unmodified enzyme, a 105-fold reduction. “5′ to 3′ exonuclease activity” is also an enzymatic activity well known in the art. This activity is often associated with DNA polymerases, such as E. coli Poll and Taq DNA polymerase. A “polymerase substantially reduced in 5′ to 3′ exonuclease activity” is defined herein as either (1) a mutated or modified polymerase that has about or less than 10%, or preferably about or less than 1%, of the 5′ to 3′ exonuclease activity of the corresponding unmutated, wild-type enzyme, or (2) a polymerase having 5′ to 3′ exonuclease specific activity which is less than about 1 unit mg protein, or preferably about or less than 0.1 units/mg protein. Both of the 3′ to 5′ and 5′ to 3′ exonuclease activities can be observed on sequencing gels. Active 5′ to 3′ exonuclease activity will produce nonspecific ladders in a sequencing gel by removing nucleotides from the 5′-end of the growing primers. 3′ to 5′ exonuclease activity can be measured by following the degradation of radiolabeled primers in a sequencing gel. Thus, the relative amounts of these activities, e.g., by comparing wild-type and mutant or modified polymerases, can be determined with no more than routine experimentation.

The nucleic acid polymerases used in the present invention may be mesophilic or thermophilic, and are preferably thermophilic. Preferred mesophilic DNA polymerases include T7 DNA polymerase, T5 DNA polymerase, Klenow fragment DNA polymerase, DNA polymerase III and the like. Preferred thermostable DNA polymerases that may be used in the invention include Taq, Tne, Tma, Pfu, Tfl, Tth, Stoffel fragment, VENT® and DEEPVENT® DNA polymerases, and mutants, variants and derivatives thereof (U.S. Pat. No. 5,436,149; U.S. Pat. No. 4,889,818; U.S. Pat. No. 4,965,188; U.S. Pat. No. 5,079,352; U.S. Pat. No. 5,614,365; U.S. Pat. No. 5,374,553; U.S. Pat. No. 5,270,179; U.S. Pat. No. 5,047,342; U.S. Pat. No. 5,512,462; U.S. Pat. No. 6,015,668; U.S. Pat. No. 5,939,301; U.S. Pat. No. 5,948,614; U.S. Pat. No. 5,912,155; WO 97/09451; WO 98/35060; WO 92/06188; WO 92/06200; WO 96/10640; Barnes, W. M., Gene 112:29-35 (1992); Lawyer, F. C., et al., PCR Meth. Appl. 2:275-287 (1993); Flaman, J.-M, et al., Nucl. Acids Res. 22(15):3259-3260 (1994)). For amplification of long nucleic acid molecules (e.g., nucleic acid molecules longer than about 3-5 Kb in length), at least two DNA polymerases (one substantially lacking 3′ exonuclease activity and the other having 3′ exonuclease activity) are typically used. See U.S. Pat. No. 5,436,149; U.S. Pat. No. 5,512,462; Barnes, W. M., Gene 112:29-35 (1992), the disclosures of which are incorporated herein in their entireties. Examples of DNA polymerases substantially lacking in 3′ exonuclease activity include, but are not limited to, Taq, Tne(exo⁻), Tma(exo⁻), Pfu(exo⁻), Pwo(exo⁻) and Tth DNA polymerases, and mutants, variants and derivatives thereof.

DNA polymerases for use in the present invention may be obtained commercially, for example, from Invitrogen Corporation (Life Technologies Division) (Rockville, Md.), Pharmacia (Piscataway, N.J.), Sigma (St. Louis, Mo.) and Boehringer Mannheim Biochemicals. Preferred DNA polymerases for use in the present invention include Tsp DNA polymerase from Invitrogen Corporation (Life Technologies Division).

Enzymes for use in the compositions, methods and kits of the invention include any enzyme having reverse transcriptase activity. Such enzymes include, but are not limited to, retroviral reverse transcriptase, retrotransposon reverse transcriptase, hepatitis B reverse transcriptase, cauliflower mosaic virus reverse transcriptase, bacterial reverse transcriptase, Tth DNA polymerase, Taq DNA polymerase (Saiki, R. K., et al., Science 239:487-491 (1988); U.S. Pat. Nos. 4,889,818 and 4,965,188), Tne DNA polymerase (WO 96/10640), Tma DNA polymerase (U.S. Pat. No. 5,374,553) and mutants, fragments, variants or derivatives thereof (see, e.g., U.S. Pat. Nos. 5,948,614 and 6,015,668, which are incorporated by reference herein in their entireties). As will be understood by one of ordinary skill in the art, modified reverse transcriptases and DNA polymerases having RT activity may be obtained by recombinant or genetic engineering techniques that are well-known in the art. Mutant reverse transcriptases or polymerases can, for example, be obtained by mutating the gene or genes encoding the reverse transcriptase or polymerase of interest by site-directed or random mutagenesis. Such mutations may include point mutations, deletion mutations and insertional mutations. Preferably, one or more point mutations (e.g., substitution of one or more amino acids with one or more different amino acids) are used to construct mutant reverse transcriptases or polymerases for use in the invention. Fragments of reverse transcriptases or polymerases may also be obtained by deletion mutation by recombinant techniques that are well-known in the art, or by enzymatic digestion of the reverse transcriptase(s) or polymerase(s) of interest using any of a number of well-known proteolytic enzymes.

Suitable enzymes for use in the invention include those that are reduced or substantially reduced in RNase H activity. Such enzymes that are reduced or substantially reduced in RNase H activity may be obtained by mutating the RNase H domain within the reverse transcriptase of interest, preferably by one or more point mutations, one or more deletion mutations, and/or one or more insertion mutations as described above. By an enzyme “substantially reduced in RNase H activity” is meant that the enzyme has less than about 30%, less than about 25%, less than about 20%, more preferably, less than about 15%, less than about 10%, less than about 7.5%, or less than about 5%, and most preferably, less than about 5% or less than about 2%, of the RNase H activity of the corresponding wildtype or RNase H⁺ enzyme such as wildtype Moloney Murine Leukemia Virus (M-MLV), Avian Myeloblastosis Virus (AMV) or Rous Sarcoma Virus (RSV) reverse transcriptases. The RNase H activity of any enzyme may be determined by a variety of assays, such as those described, for example, in U.S. Pat. No. 5,244,797, Kotewicz, M. L., et al., Nucl. Acids Res. 16:265 (1988), Gerard, G. F., et al., FOCUS 14(5):91 (1992), and U.S. Pat. Nos. 5,668,005 and 6,063,608, the disclosures of all of which are fully incorporated herein by reference.

Polypeptides having reverse transcriptase activity for use in the invention may be obtained commercially, for example from Invitrogen Corporation (Life Technologies Division) (Rockville, Md.), Pharmacia (Piscataway, N.J.), Sigma (Saint Louis, Mo.) or Boehringer Mannheim Biochemicals (Indianapolis, Ind.). Alternatively, polypeptides having reverse transcriptase activity may be isolated from their natural viral or bacterial sources according to standard procedures for isolating and purifying natural proteins that are well-known to one of ordinary skill in the art (see, e.g., Houts, G. E., et al., J. Virol. 29:517 (1979)). In addition, the polypeptides having reverse transcriptase activity may be prepared by recombinant DNA techniques that are familiar to one of ordinary skill in the art (see, e.g., Kotewicz, M. L., et al., Nucl. Acids Res. 16:265 (1988); Soltis, D. A., and Skalka, A. M., Proc. Natl. Acad. Sci. USA 85:3372-3376 (1988)).

Suitable polypeptides having reverse transcriptase activity for use in the invention include M-MLV reverse transcriptase, RSV reverse transcriptase, AMV reverse transcriptase, Rous Associated Virus (RAV) reverse transcriptase, Myeloblastosis Associated Virus (MAV) reverse transcriptase and Human Immunodeficiency Virus (HIV) reverse transcriptase, and others described in WO 98/47921 and derivatives, variants, fragments or mutants thereof, and combinations thereof. In a further preferred embodiment, the reverse transcriptases are reduced or substantially reduced in RNase H activity, and are most preferably selected from the group consisting of M-MLV H⁻ reverse transcriptase, RSV H⁻ reverse transcriptase, AMV H⁻ reverse transcriptase, RAV H⁻ reverse transcriptase, MAV H⁻ reverse transcriptase and HIV H⁻ reverse transcriptase, and derivatives, variants, fragments and mutants thereof, and combinations thereof. Reverse transcriptases of particular interest include AMV RT and M-MLV RT, and more preferably, AMV RT and M-MLV RT having reduced or substantially reduced RNase H activity (preferably, AMV RT αH⁻/BH⁺ and M-MLV RT H⁻). The most preferred reverse transcriptases for use in the invention include SuperScript™, SuperScript™II, ThermoScript™ and ThermoScript™II available from Invitrogen Corporation (Life Technologies Division) See, generally, WO 98/47921 and U.S. Pat. Nos. 5,244,797, 5,668,005 and 6,063,608, the entire contents of each of which are herein incorporated by reference.

Hairpin. As used herein, the term “hairpin” is used to indicate the structure of an oligonucleotide in which one or more portions of the oligonucleotide form base pairs with one or more other portions of the oligonucleotide. When the two portions are base paired to form a double-stranded portion of the oligonucleotide, the double-stranded portion may be referred to as a stem. Thus, depending on the number of complementary portions used, a number of stems (preferably about 1 to about 10) may be formed.

In some embodiments, the primers may be modified such that they assume a hairpin structure. This may be accomplished by adding one or more bases to the 5′-termini of the oligonucleotide wherein the bases are selected to be complementary to the bases at the 3′-termini of the oligonucleotide. In some preferred embodiments, at least one to about 20 contiguous nucleotides are added to the 5′-end of the oligonucleotide that are complementary to the at least one to 20 contiguous nucleotides present in the 3′-end of the oligonucleotide. In a preferred embodiment, from one to about 10 nucleotides are added to the 5′-end of the oligonucleotide, the nucleotides selected such that they are complementary to the at least one to about 10 contiguous nucleotides present in the 3′-end of the oligonucleotide. In another preferred embodiment, from one to about 5 nucleotides are added to the 5′-end of the oligonucleotide, the nucleotides selected such that they are complementary to the at least one to about 5 contiguous nucleotides present in the 3′-end of the oligonucleotide.

Additionally, formation of the one or more stems preferably allows formation of one or more loop structures in the hairpin molecule. In one aspect, any one or more of the loop structures may be cut or nicked at one or more sites within the loop or loops but, preferably, at least one loop is not so cut or nicked. The sequence of the oligonucleotide may be selected so as to vary the number of nucleotides which base pair to form the stem from about 3 nucleotides to about 100 or more nucleotides, from about 3 nucleotides to about 50 nucleotides, from about 3 nucleotides to about 25 nucleotides, and from about 3 to about 10 nucleotides. In addition, the sequence of the oligonucleotide may be varied so as to vary the number of nucleotides which do not form base pairs from 0 nucleotides to about 100 or more nucleotides, from 0 nucleotides to about 50 nucleotides, from 0 nucleotides to about 25 nucleotides or from 0 to about 10 nucleotides. The two portions of the oligonucleotide which base pair may be located anywhere or at any number of locations in the sequence of the oligonucleotide. In some embodiments, one base pairing portion of the oligonucleotide may include the 3′-termini of the oligonucleotide. In some embodiments, one base pairing-portion may include the 5′-termini of the oligonucleotide. In some embodiments, one base pairing portion of the oligonucleotide may include the 3′-termini while the other base pairing portion may include the 5′-termini and, when base paired, the stem of the oligonucleotide is blunt ended. In other embodiments, the location of the base pairing portions of the oligonucleotide may be selected so as to form a 3′-overhang and/or a 5′-overhang and/or may be selected so that neither the 3′- nor the 5′-most nucleotides are involved in base pairing.

Hairpin version of the oligonucleotide primers can be constructed by adding bases to the 5′-end of the primer sequence that are complementary to the 3′-end of the oligonucleotide, for example. Typically, the number of bases added to the 5′-end is selected such that the oligonucleotide forms a hairpin at temperatures below the annealing temperature and assumes a linear form at or near the annealing temperature. Those skilled in the art can readily determine the number of nucleotides to be added to the 5′-end of the primer so as to control the temperature at which the primer assumes a linear form. It is not necessary that the oligonucleotides of the invention be entirely converted to linear form at the annealing temperature; those skilled in the art will appreciate that the oligonucleotides of the present invention may be capable of reversibly melting and self-reannealing (i.e., breathing). So long as the sequences of the oligonucleotides of the invention are selected such that a sufficient number of oligonucleotides are available to prime the extension, amplification, etc. at the annealing temperature, the sequences are suitable for use in the present invention whether or not some of the oligonucleotides remain in a hairpin form at the annealing temperature. The number of nucleotides that may be added may be from about 3 nucleotides to about 25 nucleotides, or from about 3 nucleotides to about 20 nucleotides, or from about 3 nucleotides to about 15 nucleotides, or from about 3 nucleotides to about 10 nucleotides, or from about 3 nucleotides to about 7 nucleotides. In some preferred embodiments, from about 5 to about 8 nucleotides may be added to the 5′-end of the primer oligonucleotide in order to form the hairpin oligonucleotides of the present invention.

Hybridization. As used herein, the terms “hybridization” and “hybridizing” refer to the pairing of two complementary single-stranded nucleic acid molecules (RNA and/or DNA) to give a double-stranded molecule. As used herein, two nucleic acid molecules may be hybridized, although the base pairing is not completely complementary. Accordingly, mismatched bases do not prevent hybridization of two nucleic acid molecules provided that appropriate conditions, well-known in the art, are used.

Incoriorating. The term “incorporating” as used herein means becoming a part of a DNA or RNA molecule or primer.

Nucleotide. As used herein “nucleotide” refers to a base-sugar-phosphate combination. Nucleotides are monomeric units of a nucleic acid sequence (DNA and RNA). The term nucleotide includes mono-, di- and triphosphate forms of deoxyribonucleosides and ribonucleosides and their derivatives. The term nucleotide particularly includes deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well-known techniques. Detectable labels include, for example, radioactive isotopes, fluorescent labels, chemiluminescent labels, bioluminescent labels and enzyme labels.

Oligonucleotide. As used herein, “oligonucleotide” refers to a synthetic or biologically produced molecule comprising a covalently linked sequence of nucleotides or derivatives thereof. Such nucleotides may be joined by a phosphodiester bond between the 3′ or 5′ position of the pentose of one nucleotide, for example, and the 5′ or 3′ position of the pentose of the adjacent nucleotide, for example. Bonds can also occur between 3′ positions and 5′ positions and between any other at least two positions.

Oligonucleotide, as used herein, includes natural nucleic acid molecules (i.e., DNA and RNA) as well as non-natural or derivative molecules such as peptide nucleic acids, phosphothioate containing nucleic acids, phosphonate containing nucleic acids and the like. In one embodiment, oligonucleotides of the invention may comprise 5-100 nucleotides (e.g. 5-10, 15-20, 25-30, 35-40, 45-50, 55-60, 65-70, 75-80, 85-90, 95-100 etc. nucleotides), preferably, 6 nucleotides. In addition, oligonucleotides of the present invention may contain modified or non-naturally occurring sugar residues (i.e., arabainose) and/or modified base residues as described below. Oligonucleotide encompasses derivative molecules such as nucleic acid molecules comprising various natural nucleotides, derivative nucleotides, nucleotide analogues, modified nucleotides or combinations thereof.

Oligonucleotides can be DNA or RNA or chimeric mixtures or derivatives or modified versions thereof. In addition to being labeled with a detectable moiety, oligonucleotides can be modified at the base moiety, sugar moiety, or phosphate backbone, and may include other appending groups or labels. Further, oligonucleotides can be any suitable size, e.g., in the range of 10-100 or 10-80 nucleotides. Some suitable oligonucleotides are in the range of 11-40 nucleotides or 17-25 nucleotides, although oligonucleotides may be longer or shorter depending upon the need.

Oligonucleotides may be DNA, RNA, and modified nucleic acid molecules such as phosphorothioates, protein nucleic acids (PNA) and locked nucleoside analogs (LNA). Such modifications may be at or near the 3′- and/or 5′-termini of the nucleic acid molecules and/or within the nucleic acid molecule.

Modified oligonucleotides of the invention may have one or more modifications at one or more positions within the oligonucleotide(s) and/or at or near the 3′- and/or 5′-termini. In one embodiment, the oligonucleotide(s) may be modified on one of the two 3′- or 5′-most terminal nucleotides, one of the five 3′- or 5′-most terminal nucleotides, one of the ten 3′- or 5′-most terminal nucleotides, one of the fifteen 3′- or 5′-most terminal nucleotides, or one of the twenty 3′- or 5′-most terminal nucleotides. Oligonucleotide(s) may be modified at the second base from the 3′- or 5′-termini, at the third base from the 3′- or 5′-termini, at the fourth base from the 3′- or 5′-termini, at the sixth base from the 3′- or 5′-termini, or up to the twentieth base from the 3′- or 5′-termini.

Oligonucleotides suitable for the invention may be immobilized on a solid support. The methods of immobilizing labeled oligonucleotides which are quenched (by the methods of the invention) provide a homogenous method for detection of various nucleic acids. In a preferred embodiment, target DNA or RNA from a sample can be hybridized to an immobilized oligonucleotide and the change in the detectable label on the iminobilized oligonucleotide used as an indication of the presence or absence of a particular gene or sequence in the sample. Thus, the immobilized oligonucleotide can function as a probe. By immobilizing the oligonucleotide, the target nucleic acid does not need to be copied (which can result in missed sequences due to inefficient copying) or labeled. The change in the oligonucleotide detectable label can be due to hybridization or hybridization followed by enzymatic extension of the immobilized oligonucleotide. Alternatively, the enzymatic extension may result in the amplification of the nucleic acid target locally at the position of the immobilized oligonucleotide.

Oligonucleotides can be modified to enhance specificity and to reduce primer dimmer formation; e.g., as described in US Patent Publication No. 2003/0165859. Such primers can be used to enhance the specificity of amplification and/or synthesis (e.g., reduce mis-priming) and/or hybridization reactions. Reportedly, primers that form hairpin structures at temperatures around the annealing temperature of the PCR reaction or sequestration of the 3′-end of the primer makes the primers less capable of mis-priming to the target nucleic acid molecule. This increase in specificity reportedly is not dependent upon the particular target nucleic acid template and was observed with a variety of templates. The increase in specificity was reported to be particularly advantageous for the amplification of templates that are difficult to amplify and that produce low amounts or none of the desired amplification product in PCR reactions.

In addition to hairpin structures, any structure that sequesters the 3′-end of the oligonucleotide primer may be used to practice the present invention. For example, the 5′-portion of the oligonucleotide primers of the present invention may be provided with a sequence that is capable of forming a duplex such that the 3′-end interacts with the duplex to form a triplex. In general, any primer sequence that reversibly involves the 3′-portion of the primer in a stable structure that is not capable of annealing to the template DNA while in that structure may be used to practice the present invention. In some cases, it is useful to use an oligonucleotide complementary to the primer, so as to sequester the 3′-end of the primer. Complementary oligonucleotides may be provided with a 5′-overhanging region which may be designed to include self-complementary regions capable of forming hairpins. It is not necessary that the entire 3′-portion of the primer be sequestered, so long as the portion not sequestered is not capable of mis-priming the nucleic acid template, it is sufficient to practice the present invention.

Regarding hairpin structures, when a primer is in hairpin conformation, the 3′-end of the primer is base paired with the 5′-segment and thus, is less available for mispriming or primer-dimer formation. Modification at or near the 5′-termini of a hairpin primer reportedly can prevent primer-dimer formation.

An alternative method of minimizing primer-dimer formation while using hairpin primers is to make oligonucleotides with the 3′-end extended by 1 or 2 nucleotides that are not complementary to each other. Another alternative to primer-dimer reduction does not prevent primer-dimer formation, but makes them invisible. That is, by labeling one primer with a reporter and another with a quencher close to their 3′-ends, one causes quenching of fluorescence to occur. The fluorescence of the real amplicon will not be effected as soon as a nucleotide sequence longer than about 20 nucleotides separates the primers.

Thus, for an increase in specificity for nucleic acid amplification or synthesis and/or for decreased or reduced mis-annealing of primers (mis-priming) during nucleic acid synthesis or amplification, the oligonucleotides of the invention may be: (1) in hairpin conformation or otherwise configured so as to sequester or block the 3′-end of the oligonucleotide primer (for example by hybridizing a sequence at or near such 3′-termini); (2) modified at or near the 5′-termini; and/or (3) combinations of (1) and (2).

Oligonucleotides (labeled, unlabeled, hairpin, modified, or unmodified or any combination thereof) have use in nucleic acid amplification, synthesis or hybridization reactions (e.g., as primers) to detect or measure a nucleic acid product of the amplification or synthesis or hybridization reaction, thereby detecting or measuring a target nucleic acid in a sample that is complementary to all or a portion of a primer sequence. The oligonucleotides may be used in any amplification reaction including PCR, 5-RACE, Anchor PCR, “one-sided PCR,” LCR, NASBA, SDA, RT-PCR, real-time PCR, quantitative PCR, quantitative RT-PCR, and other amplification systems known in the art including in a universal primer format.

Oligonucleotides may be labeled internally, and/or, at or near the 3′- and/or 5′-termini or may be unlabeled. In one embodiment, the oligonucleotide(s) may be labeled on one of the two 3′- or 5′-most terminal nucleotides, one of the five 3′- or 5′-most terminal nucleotides, one of the ten 3′- or 5′-most terminal nucleotides, one of the fifteen 3′- or 5′-most terminal nucleotides, or one of the twenty 3′- or 5′-most terminal nucleotide. In a specific embodiment, the oligonucleotide(s) may be labeled at the second base from the 3′- or 5′-termini, at the third base from the 3′- or 5′-termini, at the fourth base from the 3′- or 5′-termini, at the sixth base from the 3′- or 5′-termini, or up to the twentieth base from the 3′- or 5′-termini.

When labeled, oligonucleotides may contain one or multiple labels (which may be the same or different). Oligonucleotides may be used as primers and/or probes. Oligonucleotides can be labeled with any moiety that undergoes a detectable change in any observable property upon hybridization and/or extension. In a preferred embodiment, the label is a fluorescent moiety and the label undergoes a detectable change in one or more fluorescent properties. Such properties include, but are not limited to, fluorescent intensity, fluorescent polarization, fluorescent lifetime and quantum yield of fluorescence.

Primer. As used herein, “primer” refers to a synthetic or biologically produced single-stranded oligonucleotide that is extended by covalent bonding of nucleotide monomers during amplification or polymerization of a nucleic acid molecule. Nucleic acid amplification often is based on nucleic acid synthesis by a nucleic acid polymerase or reverse transcriptase. Many such polymerases or reverse transcriptases require the presence of a primer that can be extended to initiate such nucleic acid synthesis. A primer is typically 11 bases or longer; most preferably, a primer is 17 bases or longer, although shorter or longer primers may be used depending on the need. As will be appreciated by those skilled in the art, the oligonucleotides of the invention may be used as one or more primers in various extension, synthesis or amplification reactions.

Probe. As used herein, “probe” refers to synthetic or biologically produced nucleic acids (DNA or RNA) which, by design or selection, contain specific nucleotide sequences that allow them to hybridize, under defined stringencies, specifically (i.e., preferentially) to target nucleic acid sequences. As will be appreciated by those skilled in the art, the oligonucleotides of the present invention may be used as one or more probes and preferably may be used as probes for the detection or quantification of nucleic acid molecules.

Substantially less extendable. As used herein, “substantially less extendable” is used to characterize an oligonucleotide that is inefficiently extended or not extended in an extension and/or amplification reaction when the 3′-most nucleotide of the oligonucleotide is not complementary to the corresponding base of a target/template nucleic acid. Preferably, an oligonucleotide is substantially less extendable as a result of the presence of a specificity enhancing group on the oligonucleotide. In this event, an oligonucleotide is substantially less extendable when the oligonucleotide is not extended or is extended by a lesser amount and/or at a slower rate than an oligonucleotide lacking the specificity-enhancing group, but having an otherwise identical structure. Those skilled in the art can readily determine if an oligonucleotide is substantially less extendable by conducting an extension reaction using an oligonucleotide containing a specificity enhancing group and comparing the extension to the extension of an oligonucleotide of the same structure, but lacking the specificity enhancing group. Under identical extension conditions, (e.g., melting temperature and time, annealing temperature and time, extension temperature and time, reactant concentrations and the like), a substantially less extendable oligonucleotide will produce less extension product when the 3′-most nucleotide of the oligonucleotide is not complementary to the corresponding nucleotide on a target/template nucleic acid than will be produced by an oligonucleotide lacking a specificity enhancing group, but having an otherwise identical structure. Alternatively, one skilled in the art can determine if an oligonucleotide is substantially less extendable by conducting allele specific PCR with a first set of oligonucleotides at least one of which comprises one or more specificity enhancing groups and with a second set of oligonucleotides lacking specificity enhancing groups, but otherwise of identical structure to those of the first set. Then, a determination is separately made for each set of primers of the difference in the amount of product made and/or the rate at which the product is made with the oligonucleotide having the 3′-nucleotide complementary to the corresponding nucleotide on a target/template nucleic acid to the amount of product made and/or the rate at which the product is made with an oligonucleotide having the 3′-nucleotide not complementary to the corresponding nucleotide on a target/template nucleic acid. Substantially less extendable oligonucleotides will produce a larger difference in amount of product made and/or rate at which product is made between 3′-complementary and 3′-not-complementary oligonucleotides. Preferably, the difference in the amount of product made and/or rate at which product is made using oligonucleotides containing specificity enhancing groups will be between from about 1.1 fold to about 1000 fold larger than the difference obtained using primers lacking specificity enhancing groups, or from about 1.1 fold to about 500 fold larger, or from about 1.1 fold to about 250 fold larger, or from about 1.1 fold to about 100 fold larger, or from about 1.1 fold to about 50 fold larger, or from about 1.1 to about 25 fold larger, or from about 1.1 to about 10 fold larger, or from about 1.1 fold to about 5 fold or from about 1.1 fold to about 2 fold larger. The amount of product can be determined using any methodology known to those of skill in the art, for example, by running the product on an agarose gel and staining with ethidium bromide and comparing to known amounts of similarly treated nucleic acid standards. The amount of product may be determined at any convenient time point in allele specific PCR. One convenient way to compare the rate of formation of product is to compare the number of cycles required to form a specified amount of product in PCR. A determination is separately made for each set of primers of the difference between the number of cycles required to make a given amount of product with the oligonucleotide having the 3′-nucleotide complementary to the corresponding nucleotide on a target/template nucleic acid and the number of cycles required to make the same amount of product with an oligonucleotide having the 3′-nucleotide not complementary to the corresponding nucleotide on a target/template nucleic acid. Substantially less extendable oligonucleotides will produce a larger difference in the number of cycles required to produce a specified amount of product between 3′-complementary and 3′-not-complementary oligonucleotides. The amount of product made can be determined using any means known to those skilled in the art, for example, by determining the fluorescence intensity of a labeled product using a thermocycler adapted to perform real time fluorescence detection. Preferably the difference between the number of cycles required to make a specified amount of product using oligonucleotides containing specificity enhancing groups will be between from about 1.05 fold to about 100 fold larger than the difference obtained using primers lacking specificity enhancing groups, or from about 1.05 fold to about 50 fold larger, or from about 1.05 fold to about 25 fold larger, or from about 1.05 fold to about 10 fold larger, or from about 1.05 fold to about 5 fold larger, or from about 1.05 to about 2.5 fold larger, or from about 1.05 to about 1.5 fold larger, or from about 1.05 fold to about 1.2 fold larger.

Support. As used herein a “support” may be any material or matrix suitable for attaching the oligonucleotides of the present invention or target/template nucleic acid sequences. Such oligonucleotides and/or sequences may be added or bound (covalently or non-covalently) to the supports of the invention by any technique or any combination of techniques well-known in the art. Supports of the invention may comprise nitrocellulose, diazocellulose, glass, silicon, polystyrene (including microtitre plates), polyvinylchloride, polypropylene, polyethylene, dextran, Sepharose®, agar, starch, nylon or any other material that allows for the immobilization of nucleic acids. Supports of the invention may be in any form or configuration including, but not limited to, a flat surface, beads, filters, membranes, sheets, frits, plugs, columns, microspheres, fibers (e.g., optical fibers) and the like. Solid supports may also include multi-well tubes (such as microtitre plates) such as 12-well plates, 24-well plates, 48-well plates, 96-well plates, and 384-well plates. Preferred beads are made of glass, latex or a magnetic material (magnetic, paramagnetic or superparamagnetic beads). When using solid a support, labeled oligonucleotide may be immobilized or added in solution (in the latter case, other components of the detection mixture will be immobilized).

Any number of different sequences can be immobilized onto a support into any number of distinct regions to detect one or more sequences, including, but not limited to, nucleic acid target sequences.

In a preferred aspect, methods of the invention may be used in conjunction with arrays of nucleic acid molecules (RNA or DNA). Arrays of nucleic acid template/target or arrays of oligonucleotides of the invention are both contemplated in the methods of the invention. Such arrays may be formed on microplates, glass slides or standard blotting membranes and may be referred to as microarrays or gene-chips depending on the format and design of the array. Uses for such arrays include gene discovery, gene expression profiling and genotyping (SNP analysis, pharmacogenomics and toxicogenetics).

Synthesis and use of nucleic acid arrays and, generally, attachment of nucleic acids to supports have been described (see, for example, U.S. Pat. No. 5,436,327, U.S. Pat. No. 5,800,992, U.S. Pat. No. 5,445,934, U.S. Pat. No. 5,763,170, U.S. Pat. No. 5,599,695 and U.S. Pat. No. 5,837,832). An automated process for attaching various reagents to positionally defined sites on a substrate is provided in U.S. Pat. No. 5,143,854 and U.S. Pat. No. 5,252,743.

Essentially, any conceivable support may be employed in the invention. The support may be biological, nonbiological, organic, inorganic, or a combination of any of these, existing as particles, strands, precipitates, gels, sheets, tubing, spheres, containers, capillaries, pads, slices, films, plates, slides, etc. The support may have any convenient shape, such as a disc, square, sphere, circle, etc. The support is preferably flat, but may take on a variety of alternative surface configurations. For example, the support may contain raised or depressed regions on which one or more methods of the invention may take place. The support and its surface preferably form a rigid support on which to carry out the reactions described herein. The support and its surface are also chosen to provide appropriate light-absorbing characteristics. For instance, the support may be a polymerized Langmuir Blodgett film, functionalized glass, Si, Ge, GaAs, GaP, SiO₂, SIN₄, modified silicon, or any one of a wide variety of gels or polymers such as (poly)tetrafluoroethylene, (poly)vinylidenedifluoride, polystyrene, polycarbonate, or combinations thereof. Other support materials will be readily apparent to those of skill in the art upon review of this disclosure. In a preferred embodiment, the support is flat glass or single-crystal silicon.

Target molecule. As used herein, “target molecule” refers to a nucleic acid molecule to which a particular primer or probe is capable of preferentially hybridizing.

Target sequence. As used herein, “target sequence” refers to a nucleic acid sequence within the target molecules to which a particular primer or probe is capable of preferentially hybridizing.

Template. The term “template” as used herein refers to a double-stranded or single-stranded molecule which is to be amplified, synthesized or sequenced. In the case of a double-stranded DNA molecule, denaturation of its strands to form a first and a second strand is preferably performed to amplify, sequence or synthesize these molecules. A primer, complementary to a portion of a template is hybridized under appropriate conditions and the polymerase (DNA polymerase or reverse transcriptase) may then synthesize a nucleic acid molecule complementary to said template or a portion thereof. The newly synthesized molecule, according to the invention, may be equal or shorter in length than the original template. Mismatch incorporation during the synthesis or extension of the newly synthesized molecule may result in one or a number of mismatched base pairs. Thus, the synthesized molecule need not be exactly complementary to the template. The template can be an RNA molecule, a DNA molecule or an RNA/DNA hybrid molecule. A newly synthesized molecule may serve as a template for subsequent nucleic acid synthesis or amplification.

Thermostable. As used herein, “thermostable” refers to a polymerase (RNA, DNA or RT) which is resistant to inactivation by heat. DNA polymerases synthesize the formation of a DNA molecule complementary to a single-stranded DNA template by extending a primer in the 5′-to-3′ direction. This activity for mesophilic DNA polymerases may be inactivated by heat treatment. For example, T5 DNA polymerase activity is totally inactivated by exposing the enzyme to a temperature of 90° C. for 30 seconds. As used herein, a thermostable DNA polymerase activity is more resistant to heat inactivation than a mesophilic DNA polymerase. However, a thermostable DNA polymerase does not mean to refer to an enzyme which is totally resistant to heat inactivation and thus, heat treatment may reduce the DNA polymerase activity to some extent. A thermostable DNA polymerase typically will also have a higher optimum temperature than mesophilic DNA polymerases.

Vector. As used herein, is a DNA that is able to replicate or be replicated in vitro or in a host cell or that provides a useful biological or biochemical property to an inserted gene. Examples include plasmids, phages, and other DNA sequences. A Vector can have one or more restriction endonuclease recognition sites at which the DNA sequences can be cut in a determinable fashion without loss of an essential biological function of the vector, and into which a DNA fragment can be spliced in order to bring about its replication and cloning. Vectors can further provide primer sites, e.g., for PCR, transcriptional and/or translational initiation and/or regulation sites, recombinational signals, replicons, Selectable markers, etc. The cloning vector can further contain a Selectable marker suitable for use in the identification of cells transformed with the cloning vector. Any number of hosts may be used to express the present invention; including prokaryotic and eukaryotic cells. Host cells that may be used are those well known in the art.

Other terms used in the fields of recombinant DNA technology and molecular and cell biology as used herein will be generally understood by one of ordinary skill in the applicable arts.

Use of Sequence Nonspecific Double-Stranded DNA Binding Proteins in Nucleic Acid Amplification

In one embodiment, sequence nonspecific double-stranded DNA binding proteins are used to enhance the efficiency, specificity and/or sensitivity of nucleic acid amplification reactions (e.g., PCR-based amplification reactions). Such amplification reactions include PCR, 5-RACE, Anchor PCR, “one-sided PCR,” LCR, NASBA, SDA, linear PCR, mutagenic PCR, allele specific amplification, RT-PCR, real-time PCR (qPCR), quantitative PCR, quantitative RT-PCR, and other amplification systems known in the art including in a universal primer format.

One exemplary nonspecific double-stranded DNA binding protein is SsO7d, derived from the thermophile Sulfolobus solfataricus. The materials and methods described herein certainly are not limited to the use of SsO7d. Other nonspecific double-stranded DNA binding proteins that exhibit at least about 80% sequence identity at the amino acid level also can be used in the disclosed materials and methods, including proteins (designated by GenBank accession numbers) shown in Table 1.

TABLE 1

| Protein | Organism | % identity |
|---|---|---|
| NP_343422.1 | Sulfolobus solfataricus | 100 |
| NP_343889 | Sulfolobus solfataricus | 98 |
| S69085 | Sulfolobus solfataricus | 92 |
| A27749 | Sulfolobus acidocaldarius | 84 |
| P13123 | Sulfolobus acidocaldarius | 78 |
| O59632 | Sulfolobus shibatae | 95 |
| NP_378090.1 | Sulfolobus tokodaii | 89 |

Other suitable nonspecific double-stranded binding proteins include scaffolding proteins (e.g., high mobility group (HMG), structural maintenance of chromosome (SMC) and nucleosome remodeling proteins) and histone-like proteins, including heat unstable nucleoid protein (HU), integration host factor (IHF), factor for inversion stimulation (FIS), histone-like nucleoid structuring protein (HNS), suppressor of thymidylate synthase mutant phenotype A (StpA) and DNA binding protein from starved cells (Dps).

Other proteins having sequence, structure and/or domains related to SsO7d are also contemplated for use in the present methods. Such proteins can be identified using the method of Bailey et al. (Proceedings of the Third International Conference of Computational Molecular Biology (RECOMB99), pp. 10-14, S. Istrail et al., Eds., ACM, New York, 1999). For example, a query was filtered for low complexity regions using the “seg” program using the following settings:

Family library: SCOP v1.50 domain sequences with 40% identity

Pairwise search: Smith-Waterman on the Compugen BioXL-P

Scoring matrix: Biosum 62

Gap Opening penalty: 11

Gap extension penalty: 1

The query results include the following protein families, all members of which may be suitable for use in nucleic amplification reactions (e.g., in reducing the background fluorescence of hairpin primers): chromo-domain like (“histone-like” proteins from archaea, chromo domain); ds-RNA-binding domain-like (ds-RNA-binding domain, homologous-pairing domain of Rad52 recombinase, ribosomal S5 protein, N-terminal domain); EF-Tu/eEF-1alpha/eIF2-gamma C-terminal domain (elongation factor Tu, elongation factor eEF-1 alpha, C-terminal domain, initiation factor eIF2 gamma subunit); nucleic-acid binding proteins (anticodon-binding domain, RecG “wedge” domain, DNA helicase RuvA subunit, N-terminal domain, single strand DNA binding domain SSB, Myf domain, cold shock DNA-binding domain-like, hypothetical protein MTH1 (MT0001), insert domain, DNA ligase/mRNA capping enzyme, domain 2, phage ssDNA-binding proteins, DNA replication initiator (cdc21/cdc54) N-terminal domain, RNA polymerase subunit RBP8).

The ability of a nonspecific double-stranded DNA binding protein to enhance the efficiency, specificity and/or sensitivity of nucleic acid amplification reactions may be determined as described herein without undue experimentation, for example as described in Examples 3-5.

The present invention provides a method of determining the presence of a particular nucleotide or nucleotides at a specific position or positions in a target or template nucleic acid molecule, comprising: (a) contacting at least one target or template nucleic acid molecule having a nucleotide or nucleotides at a specific position or positions with one or more oligonucleotides of the invention, wherein at least a portion of the oligonucleotide is capable of forming base pairs (e.g., hybridizing) with at least a portion of the target or template nucleic acid molecule, said oligonucleotide preferably comprises at least one label; and a nonspecific double-stranded DNA binding protein; and (b) incubating the oligonucleotide and the nucleic acid molecule mixture under conditions sufficient to cause extension of the oligonucleotide when the 3′-most nucleotide or nucleotides of the oligonucleotide base pair with the nucleotide or nucleotides at the specific position or positions of the nucleic acid target molecule. Under such conditions, the production of an extension product indicates the presence of the particular nucleotide or nucleotides at the specific position or positions. Presence of or increased production of an extension product, as used herein, refers to the difference in the amount of amplifed DNA made using the modified oligonucleotides will be between 1 fold to about 1000 fold larger than the difference obtained using oligonucleotides lacking the modification, or from about 1 fold to about 500 fold larger, or from about 1 fold to about 250 fold larger, or from about 1 fold to about 100 fold larger, or from about 1 fold to about 50 fold larger, or from about 1 to about 25 fold larger, or from about 1 to about 10 fold larger, or from about 1 fold to about 5 fold or from about 1 fold to about 2 fold larger. The amount of product can be determined using any methodology known to those of skill in the art, for example, by running the product on an agarose gel and staining with ethidium bromide and comparing to known amounts of similarly treated nucleic acid standards.

In another aspect, there is provided a method for determining the absence of at least one particular nucleotide at a specific position or positions in a target or template nucleic acid molecule, comprising: (a) contacting at least one target nucleic acid molecule having a nucleotide or nucleotides at a specific position with an oligonucleotide of the invention, wherein at least a portion of the oligonucleotide is capable of forming base pairs (e.g., hybridizing) with at least a portion of the target nucleic acid molecule (said oligonucleotide preferably comprising at least one label); and a nonspecific double-stranded DNA binding protein; and (b) incubating the oligonucleotide and the nucleic acid molecule mixture under conditions sufficient to prevent or inhibit extension of the oligonucleotide when the 3′-most nucleotide or nucleotides of the oligonucleotide does not base pair (e.g., does not hybridize) with the nucleotide at the specific position or positions of the target nucleic acid molecule. Under such conditions, the lack of production or reduced production of an extension product indicates the absence of the particular nucleotide or nucleotides at the specific position. Lack of or reduced production of an extension product, as used herein, refers to the difference in the amount of amplified DNA made using oligonucleotides lacking modifications, or from about 1 fold to about 500 fold larger than the difference obtained using modified oligonucleotides, or from about 1 fold to about 250 fold larger, or from about 1 fold to about 100 fold larger, or from about 1 fold to about 50 fold larger, or from about 1 to about 25 fold larger, or from about 1 to about 10 fold larger, or from about 1 fold to about 5 fold larger or from about 1 fold to about 2 fold larger. In a preferred aspect, the results of the extension of the oligonucleotide in the above first method is compared to the lack or reduced level of extension of the oligonucleotide in the above second method. In a preferred aspect, the conditions in the first method are conducted such that all or a portion of the target nucleic acid molecule is amplified, while the conditions in the second method are conducted such that the target nucleic acid molecule is not amplified or amplified at a reduced level or slower rate compared to the amplified target nucleic acid molecule produced by the first method. In some embodiments, at least a portion of said oligonucleotide is hybridized to at least a portion of said nucleic acid molecule. In some embodiments, the oligonucleotide is capable of forming a hairpin. In some embodiments, the oligonucleotide is in the form of a hairpin. The conditions of incubation preferably include the presence of one or more polymerase enzymes such as Tsp DNA polymerase (available from Invitrogen Corporation (Life Technologies Division), Rockville, Md.).

The present invention provides a method of synthesizing one or more nucleic acid molecules, comprising: (a) contacting at least one target or template nucleic acid molecule with at least one oligonucleotide of the invention, wherein at least a portion of said oligonucleotide is capable of hybridizing with at least a portion of said target/template nucleic acid molecule (said oligonucleotide preferably comprises at least one specificity enhancing group and/or label); and a nonspecific double-stranded DNA binding protein; and (b) incubating the target nucleic acid and oligonucleotide mixture under conditions sufficient to cause the extension of the oligonucleotide when the 3′-most nucleotide or nucleotides of the oligonucleotide are base paired (e.g. hybridized) to said target nucleic acid molecule.

The present invention also provides a method for amplifying a double-stranded nucleic acid molecule, comprising:

(a) providing a first and second primer, wherein said first primer is complementary to a sequence within or at or near the 3′-termini of the first strand of said nucleic molecule and said second primer is complementary to a sequence within or at or near the 3′-termini of the second strand of said nucleic acid molecule;

(b) hybridizing said first primer to said first strand and said second primer to said second strand in the presence of one or more of the polymerases and a nonspecific double-stranded DNA binding protein, under conditions such that a third nucleic acid molecule complementary to all or a portion of said first strand and a fourth nucleic acid molecule complementary to all or a portion said second strand are synthesized;

(c) denaturing said first and third strand, and said second and fourth strands; and repeating the above steps one or more times, wherein one or more of the primers comprise a nucleotide modification at or near the 3′-terminal nucleotide.

In another aspect, there is provided a method of determining the presence of at least one nucleotide of interest at a specific position in a target nucleic acid molecule, comprising:

(a) contacting at least one target nucleic acid molecule having said nucleotide of interest at a specific position on a target nucleic acid molecule with at least one oligonucleotide, wherein at least a portion of the oligonucleotide is capable of forming base pairs or hybridizing with at least a portion of the target nucleic acid molecule and wherein the oligonucleotide comprises a nucleotide modification at or near the 3′-terminal nucleotide; and

(b) incubating the oligonucleotide, the target nucleic acid molecule and a nonspecific double-stranded DNA binding protein under conditions sufficient to cause extension of the oligonucleotide when the 3′-most nucleotide of the oligonucleotide base pair with the nucleotide at the specific position of the target nucleic acid molecule, wherein the presence of or increased production of an extension product indicates the presence of the particular nucleotide at the specific position.

Another embodiment is a method of determining the absence of at least one nucleotide at a specific position in a target nucleic acid molecule, comprising:

(a) contacting at least one target nucleic acid molecule having said nucleotide of interest at a specific position on the target nucleic acid molecule with at least one oligonucleotide, wherein at least one portion of the oligonucleotide is capable of forming base pairs or hybridizing with at least a portion of the target nucleic acid molecule and wherein the oligonucleotide comprises a nucleotide modification at or near the 3′-terminal nucleotide; and

(b) incubating the oligonucleotide, the target nucleic acid molecule and a nonspecific double-stranded DNA binding protein under conditions sufficient to inhibit or prevent extension of the oligonucleotide when the 3′-most nucleotide of the oligonucleotide does not substantially base pair with the nucleotide of the specific position of the target nucleic acid molecule, wherein the lack of or reduced production of an extension product indicates the absence of the particular nucleotide at the specific position.

In another aspect, the invention provides a method of determining the presence or absence of a nucleotide at a specific position in a target nucleic acid molecule, comprising:

(a) contacting at least first oligonucleotide with at least one target nucleic acid molecule under conditions sufficient to cause extension of the first oligonucleotide when the 3′-most nucleotide of the oligonucleotide base pairs with the nucleotide at the specific position of the target nucleic acid molecule, wherein said first oligonucleotide comprises a nucleotide modification at or near the 3′-terminal nucleotide;

(b) contacting at least a second oligonucleotide with at least one target nucleic acid molecule under conditions sufficient to inhibit or prevent extension of the oligonucleotide when the 3′-most nucleotide of the oligonucleotide do not substantially base pair with the nucleotide at the specific position of the target nucleic acid molecule, wherein said second oligonucleotide comprises a nucleotide modification at or near the 3′-terminal nucleotide; and

(c) comparing the level of extension or the amount of extension or presence or absence of extension product accomplished with the first oligonucleotide compared to the second oligonucleotide.

The amount of product can be determined using any methodology known to those of skill in the art, for example, by running the product on an agarose gel and staining with ethidium bromide and comparing to known amounts of similarly treated nucleic acid standards.

The present invention provides a method for synthesizing or amplifying one or more nucleic acid molecules comprising:

(a) mixing one or more nucleic acid templates or targets with one or more oligonucleotides, wherein said one or more of said oligonucleotides comprise a nucleotide modification at or near the 3′-terminal nucleotide, and a nonspecific double-stranded DNA binding protein; and

(b) incubating said mixture under conditions sufficient to synthesize or amplify one or more nucleic acid molecules complementary to all or a portion of said templates or targets.

In another aspect, the invention provides a method for synthesizing or amplifying one or more nucleic acid molecules, wherein the specificity of the nucleic acid synthesis or amplification is increased, comprising:

(a) mixing one or more nucleic acid templates or targets with one or more oligonucleotides, wherein said one or more of said oligonucleotides comprises a nucleotide modification at or near the 3′-terminal nucleotide; and a nonspecific double-stranded DNA binding protein; and

(b) incubating said mixture under conditions sufficient to synthesize or amplify one or more nucleic acid molecules complementary to all or a portion of said templates or targets, wherein the synthesis or amplification has increased specificity when compared to amplification or synthesis conducted with an oligonucleotide not modified with a nucleotide modification at or near the 3′-terminal nucleotide.

There is also provided a method for the quantitation or detection of one or more nucleic acid molecules in a sample during nucleic acid synthesis comprising:

(a) mixing one or more nucleic acid templates with one or more oligonucleotides and a nonspecific double-stranded DNA binding protein, wherein said oligonucleotides are oligonucleotides described herein or are oligonucleotides that contain one or more nucleotide analogues described herein;

(b) incubating said mixture under conditions sufficient to synthesize one or more nucleic acid molecules complementary to all or a portion of said templates, said synthesized nucleic acid molecule comprising said oligonucleotides; and

(c) detecting the presence or absence or quantifying the amount of said synthesized nucleic acid molecules by measuring the amount of nucleic acid molecules synthesized in said sample.

In another aspect, there is provided a method for quantitation or detection of one or more nucleic acid molecules in a sample during nucleic acid amplification comprising:

(a) mixing one or more nucleic acid templates with one or more oligonucleotides and a nonspecific double-stranded DNA binding protein, wherein said oligonucleotides are oligonucleotides described herein or are oligonucleotides that contain one or more nucleotide analogues described herein; and

(b) incubating said mixture under conditions sufficient to amplify one or more nucleic acid molecules complementary to all or a portion of said templates, said amplified nucleic acid molecule comprising said oligonucleotides; and

(c) detecting the presence or absence or quantifying the amount of said nucleic acid molecules by measuring the amount of nucleic acid molecules amplified in said sample.

Another embodiment is a method for amplifying a double-stranded nucleic acid molecule, comprising:

(a) providing a first and second primer, wherein said first primer is complementary to a sequence within or at or near the 3′-termini of the first strand of said nucleic molecule and said second primer is complementary to a sequence within or at or near the 3′-termini of the second strand of said nucleic acid molecule;

(b) hybridizing said first primer to said first strand and said second primer to said second strand in the presence of one or more of the polymerases and a nonspecific double-stranded DNA binding protein, under conditions such that a third nucleic acid molecule complementary to all or a portion of said first strand and a fourth nucleic acid molecule complementary to all or a portion said second strand are synthesized;

(c) denaturing said first and third strand, and said second and fourth strands; and

(d) repeating the above steps one or more times, wherein one or more of the primers comprise one or more of the nucleotide analogues of the present invention.

In another aspect, there is provided a method of determining the presence of one or more particular nucleotides at a specific position or positions in a target nucleic acid molecule, comprising:

(a) contacting at least one target nucleic acid molecule having one or more nucleotides of interest at a specific position or positions on a target nucleic acid molecule with at least one oligonucleotide, wherein at least a portion of the oligonucleotide is capable of forming base pairs or hybridizing with at least a portion of the target nucleic acid molecule and wherein the oligonucleotide is an oligonucleotide described herein or is an oligonucleotide which comprises one or more nucleotide analoguesdescribed herein; and

(b) incubating the oligonucleotide, the target nucleic acid molecule and a nonspecific double-stranded DNA binding protein under conditions sufficient to cause extension of the oligonucleotide when the 3′-most nucleotide or nucleotides of the oligonucleotide base pair with the nucleotide or nucleotides at the specific position or positions of the target nucleic acid molecule, wherein the production of an extension product indicates the presence of the particular nucleotide at the specific position.

Another embodiment is a method of determining the absence of one or more particular nucleotides at a specific position or positions in a target nucleic acid molecule, comprising:

(a) contacting at least one target nucleic acid molecule having one or more nucleotides of interest at a specific position or positions on the target nucleic acid molecule with at least one oligonucleotide, wherein at least one portion of the oligonucleotide is capable of forming base pairs or hybridizing with at least a portion of the target nucleic acid molecule and wherein the oligonucleotide is an oligonucleotide of the present invention or is an oligonucleotide which comprises one or more nucleotide analogues of the present invention; and

(b) incubating the oligonucleotide, target nucleic acid molecule and a nonspecific double-stranded DNA binding protein under conditions sufficient to inhibit or prevent extension of the oligonucleotide when the 3′-most nucleotide or nucleotides of the oligonucleotide does not substantially base pair with the nucleotide or nucleotides of the specific position or positions of the target nucleic acid molecule, wherein the lack of or reduced production of an extension product indicates the absence of the particular nucleotide at the specific position.

In another aspect, there is provided a method of determining the presence or absence of one or more particular nucleotides at a specific position or positions in a target nucleic acid molecule, comprising:

(a) contacting at least a first oligonucleotide with at least one target nucleic acid molecule and a nonspecific double-stranded DNA binding protein under conditions sufficient to cause extension of the first oligonucleotide when the 3′-most nucleotide or nucleotides of the oligonucleotide base pairs with the nucleotide or nucleotides at the specific position or positions of the target nucleic acid molecule;

(b) contacting at least a second oligonucleotide with at least one target nucleic acid molecule under conditions sufficient to inhibit or prevent extension of the oligonucleotide when the 3′-most nucleotide or nucleotides of the oligonucleotide do not substantially base pair with the nucleotide or nucleotides at the specific position or positions of the target nucleic acid molecule; and

(c) comparing the level of extension or the amount of extension product accomplished with the first oligonucleotide compared to the second oligonucleotide, wherein said first and/or second oligonucleotide is an oligonucleotide of the present invention or is an oligonucleotide which comprises one or more nucleotide analogues of the present invention.

Another embodiment is a method of determining the presence or absence of at least one particular nucleotide of interest at a specific position in a target nucleic acid molecule, comprising:

(a) providing at least one target nucleic acid molecule having said nucleotide of interest at a specific position;

(b) contacting said target nucleic acid molecule with at least one oligonucleotide, wherein at least a portion of the oligonucleotide is capable of forming base pairs or hybridizing with at least a portion of the nucleic acid molecule and wherein the oligonucleotide is an oligonucleotide of the present invention or is an oligonucleotide which comprises at least one nucleotide analogues of the present invention; and

(c) contacting the oligonucleotide and the target nucleic acid molecule with a nonspecific double-stranded DNA binding protein and a polymerase less able to extend the oligonucleotide when the 3′-most nucleotide of the oligonucleotide does not base pair with the target nucleic acid and more able to extend the oligonucleotide when the 3′-most nucleotide of the oligonucleotide base pairs with the target nucleic acid molecule; and measuring the level of extension of the oligonucleotide.

In another aspect there is provided a method for synthesizing or amplifying one or more nucleic acid molecules comprising:

(a) mixing one or more nucleic acid templates or targets, one or more oligonucleotides and a nonspecific double-stranded DNA binding protein, wherein said oligonucleotides is an oligonucleotide described herein or is an oligonucleotide which comprises one or more nucleotide analogues described herein; and

(b) incubating said mixture under conditions sufficient to synthesize or amplify one or more nucleic acid molecules complementary to all or a portion of said templates or targets.

Another embodiment is a method of detecting a single nucleotide polymorphism comprising the steps of:

(a) contacting at least a first oligonucleotide with at least one target nucleic acid molecule and a nonspecific double-stranded DNA binding protein under conditions sufficient to cause extension of the first oligonucleotide when the 3′-most nucleotide or nucleotides of the oligonucleotide base pairs with the nucleotide or nucleotides at the specific position or positions of the target nucleic acid molecule;

(b) contacting at least a second oligonucleotide with at least one target nucleic acid molecule under conditions sufficient to inhibit or prevent extension of the oligonucleotide when the 3′-most nucleotide or nucleotides of the oligonucleotide do not substantially base pair with the nucleotide or nucleotides at the specific position or positions of the target nucleic acid molecule; and

(c) comparing the level of extension or the amount of extension product or the presence of absence of extension product accomplished with the first oligonucleotide compared to the second oligonucleotide, wherein said first and/or second oligonucleotide is an oligonucleotide of the present invention or is an oligonucleotide which comprises one or more nucleotide analogues of the present invention.

### EXAMPLE 1

### Preparation of Oligonucleotides

Oligonucleotides may be prepared using any known methodology. In some preferred embodiments, oligonucleotides may be synthesized on solid supports using commercially available technology. Oligodeoxynucleotides were synthesized using DNA synthesizer-8700 (Milligen/Biosearch). Fluorescent moieties may be incorporated into the oligonucleotides of the present invention using any conventional technology and at any number of locations (e.g. at any nucleotide) within the oligonucleotide. For example, fluorescent labels may be incorporated into nucleoside phosporamidites and directly incorporated into the oligodeoxynucleotides during automated chemical synthesis. In some preferred embodiments, the modified nucleotide may be a fluorescein-dT phosphoramidite (Glen Research, cat. # 10-1056) which may be inserted into designated position during chemical synthesis of oligonucleotide. 5′-fluorescein phosphoramidite (FAM) (Glen Research, cat. # 10-5901) and 3′-TAMRA-CPG 500 (Glen Research, cat. # 20-5910) were used to add the indicated labels to the 5′- and 3′-ends, respectively, of the oligodeoxynucleotide during chemical synthesis. Alternatively, a nucleotide containing a reactive functional moiety may be incorporated into the oligonucleotide during synthesis. After the completion of the synthesis and removal of the oligonucleotide from the solid support, the reactive functional moiety may by used to couple a fluorescent moiety containing molecule to the oligonucleotide. In some preferred embodiments, the reactive functional moiety may be an amino-modified C6-dT (Glen Research, cat. # 10-1039) which may be inserted into a designated position during chemical synthesis of the oligonucleotide and used for further modification. The further modification may include the incorporation of a fluoresently labeled molecule. In some preferred embodiments, the fluorescently labeled molecule may be a 6-carboxyfluorescein succinimidyl ester (6-FAM, SE, cat. # C6164, Molecular Probes), fluorescein-5-isothiocyanate (FITC) (Molecular Probes, cat. # F-1907), 5-(6-)-carboxytetramethylrhodamine (TAMRA) succinimidyl ester (Molecular Probes), or BODIPY 530/550 succinimidyl ester (Molecular Probes).

All labeled oligonucleotides may be purified using reverse-phase HPLC, for example, on a C-18 column using a gradient of acetonitrile in 0.2 M triethyl ammonium acetate.

Oligonucleotides may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al., Nucl. Acids Res. 16:3209 (1988), and methylphosphonate oligonucleotides can be prepared by use of controlled pore glass polymer supports (Sarin et al., Proc. Natl. Acad. Sci. USA 85:7448-7451 (1988)). Oligonucleotides may also be prepared by standard phosphoramidite chemistry, or by cleavage of a larger nucleic acid fragment using non-specific nucleic acid cleaving chemicals or enzymes or site-specific restriction endonucleases. Labeled oligonucleotides may also be obtained commercially from Invitrogen Corporation (Life Technologies Division) or other oligonucleotide manufacturers.

A preferable method for synthesizing oligonucleotides is to use an automated DNA synthesizer using methods known in the art. Once the desired oligonucleotide is synthesized, it is cleaved from the solid support on which it was synthesized and treated, by methods known in the art, to remove any protecting groups present. The oligonucleotide may then be purified by any method known in the art, including extraction and gel purification. The concentration and purity of the oligonucleotide may be determined by examining the oligonucleotide that has been separated on an acrylamide gel or by measuring the optical density at 260 nm in a spectrophotometer.

Oligonucleotides may be labeled during chemical synthesis or the label may be attached after synthesis by methods known in the art. In a specific embodiment, the label moiety is a fluorophore. Suitable moieties that can be selected as fluorophores or quenchers are set forth in Table 2.

TABLE 2
4-acetamido-4′-isothiocyanatostilbene-2,2′disulfonic acid
acridine and derivatives:
acridine
acridine isothiocyanate
5-(2′-aminoethyl)aminonaphthalene-1-sulfonic acid (EDANS)
4-amino-N-3-vinylsulfonyl)phenylnaphthalimide-3,5
disulfonate (Lucifer Yellow VS)
N-(4-anilino-1-naphthyl)maleimide
anthranilamide
Brilliant Yellow
coumarin and derivatives:
7-amino-4-methylcoumarin (AMC, Coumarin 120)
7-amino-4-trifluoromethylcouluarin (Coumaran 151)
cyanosine
4′,6-diaminidino-2-phenylindole (DAPI)
5′,5″-dibromopyrogallol-sulfonephthalein (Bromopyrogallol Red)
7-diethylamino-3-(4′-isothiocyanatophenyl)-4-methylcoumarin
diethylenetriamine pentaacetate
4,4′-diisothiocyanatodihydro-stilbene-2,2′-disulfonic acid
4,4′-diisothiocyanatostilbene-2,2′-disulfonic acid
5-dimethylaminonaphthalene-1-sulfonyl chloride (DNS, dansyl chloride)
4-(4′-dimethylaminophenylazo)benzoic acid (DABCYL)
4-dimethylaminophenylazophenyl-4′-isothiocyanate (DABITC)
eosin and derivatives:

eosin
eosin isothiocyanate

erythrosin and derivatives:

erythrosin B
erythrosin isothiocyanate

ethidium
fluorescein and derivatives:
5-carboxyfluorescein (FAM)
5-(4,6-dichlorotriazin-2-yl)aminofluorescein (DTAF)
2′7′-dimethoxy-4′5′-dichloro-6-carboxyfluorescein (JOE)
fluorescein
fluorescein isothiocyanate
QFITC (XRITC)
fluorescamine
IR144
IR1446
Malachite Green isothiocyanate
4-methylumbelliferone
ortho cresolphthalein
nitrotyrosine
pararosaniline
Phenol Red
B-phycoerythrin
o-phthaldialdehyde
pyrene and derivatives:
pyrene
pyrene butyrate
succinimidyl 1-pyrene butyrate
Reactive Red 4 (Cibacron ® Brilliant Red 3B-A)
rhodamine and derivatives:
6-carboxy-X-rhodamine (ROX)
6-carboxyrhodamine (R6G)
lissamine rhodamine B sulfonyl chloride
rhodamine (Rhod)
rhodamine B
rhodamine 123
rhodamine X isothiocyanate
sulforhodamine B
sulforhodamine 101
sulfonyl chloride derivative of sulforhodamine 101 (Texas Red)
N,N,N′,N′-tetramethyl-6-carboxyrhodamine (TAMRA)
tetramethyl rhodamine
tetramethyl rhodamine isothiocyanate (TRITC)
riboflavin
rosolic acid
terbium chelate derivative

One of ordinary skill in the art can easily determine, using art-known techniques of spectrophotometry, which of the above identified fluorophores or combinations thereof can be used in accordance with the invention. Oligonucleotides are preferably modified during synthesis, such that a modified T-base is introduced into a designated position by the use of Amino-Modifier C6 dT (Glen Research), and a primary amino group is incorporated on the modified T-base, as described by Ju et al. (Proc. Natl. Acad. Sci., USA 92:4347-4351(1995)). These modifications may be used for subsequent incorporation of fluorescent dyes into designated positions of the labeled oligonucleotides.

In yet another embodiment, the labeled oligonucleotides may be further labeled with any other art-known detectable marker, including radioactive labels such as ³²p, ³¹S, ³H, and the like, or with enzymatic markers that produce detectable signals when a particular chemical reaction is conducted, such as alkaline phosphatase or horseradish peroxidase. Such enzymatic markers are preferably heat stable so as to survive the denaturing steps of the amplification or synthesis process.

Oligonucleotides may also be indirectly labeled by incorporating a nucleotide linked covalently to a hapten or to a molecule such as biotin, to which a labeled avidin molecule may be bound, or digoxygenin, to which a labeled anti-digoxygenin antibody may be bound. Oligonucleotides may be supplementally labeled during chemical synthesis or the supplemental label may be attached after synthesis by methods known in the art.

### EXAMPLE 2

### Detection of Nucleic Acids

Nucleic acids may be detected by any conventional technology. In some preferred embodiments, the nucleic acid to be detected may be a PCR product and may be detected either by agarose gel electrophoresis or by homogeneous fluorescence detection method. In this method, a fluorescent signal is generated upon the incorporation of the specifically labeled primer into the PCR product. The method does not require the presence of any specific quenching moiety or detection oligonucleotide. In some preferred embodiments, the detection oligonucleotides are capable of forming a hairpin structure and are labeled with fluorescein attached at or near to the 3′-end.

The fluorescent measurements were performed in the PCR reaction buffer using on ABI PRISM® 7700 Sequence Detector, fluorescent plate reader (TECAN) or KODAK® EDAS Digital Camera. Excitation/emission wavelengths were 490 nm/520 nm for fluorescein and 555 nm/580 nm for TAMRA. Those skilled in the art will appreciate that any nucleic acid that can be amplified by PCR may be used in the methods described herein. The selection of suitable amplification (e.g., PCR) conditions is within the purview of one of ordinary skill in the art. Those skilled in the art will realize that it may be necessary to adjust the concentrations of the nucleic acid target, primers and temperatures of the various steps in order to optimize the PCR reaction for a given target and primer. Such optimization does not entail undue experimentation.

### EXAMPLE 3

### Universal Detection Primer Format Coupled to Allele Specific PCR

The fluorescent modified oligonucleotides can be used in a format which will allow the primer to detect any gene-specific nucleotide target sequence. More specifically, fluorogenic detection primers of a design described in previous examples were used to detect the presence of PCR product in a “universal” primer format which requires three primers. The methodology requires adding a sequence tail (tail X) to the 5′-end of a gene-specific target primer used in the PCR. Tail X is non-complementary to the target. The tailed sequence is identical to the 3′-sequence of the fluorogenic detection primer. The second primer is the universal primer which is at least partially identical to tail X and labeled with a fluorescent moiety (linear or hairpin). The third primer is a regular PCR primer. If the first primer is forward, then the third primer is reverse and opposite. Thus, both forward and reverse primers and a Universal primer may be used. The forward and reverse primers may have X tails or one may have an X tail and the other may have a Y tail. The universal primer may contain the X tail sequence or two universal primers may be used wherein one contains the X tail sequence and one contains the Y tail sequence. The amplicon generated in the early cycles of PCR serves as template for the universal detection primer in the later cycles.

In this example, the gene specific sequences were designed for allele-specific PCR, but the universal detection format can be used for any target by adding a 5′ tail of appropriate sequence to the gene specific primer. PCR was performed in a 50 μl volume of reaction buffer (20 mM Tris-HCl (pH 8.4), 50 mM KCl) containing 1.5 mM MgCl₂, 0.2 mM each dNTP, 2.5 U PLATINUM® Taq DNA polymerase, 0.2 μM forward universal labeled detection primer, 0.02 μM forward allele specific tailed primer, and 0.2 μM reverse primer. Thermal cycling and fluorescent detection were performed on an ABI 7700 Sequence Detection System using a 2 minute hold at 25° C., a 2 minute hold at 94° C., and 40 cycles of 30 seconds at 94° C., 30 seconds at 55° C. and 30 seconds at 72° C., and a 2 minute hold at 25° C.

Primer sequences used were Oligo 48 for the universal detection primer, Oligo 49 for the forward allele A-specific primer, and Oligo 50 for the reverse primer. Universal format detection and allelic discrimination were equivalent using forward allele B-specific primer (Oligo 51). Other universal detection sequences (Oligos 52-58) were used in the same experiment and gave similar results (Table 3). These and other universal primer sequences were labeled with dyes other than FAM (JOE, TAMRA, ALEXA 450, ALEXA 594, HEX, and TET), giving similar results.

TABLE 3

| | |
|---|---|
| Oligo 48 5′- cta ccg ggt gtc tgt gtc tcg gTa g | (SEQ ID NO: 1) |
| Oligo 49 5′- ggg tgt ctg tgt ctc ggt aga cct ggc tat ctg tgt c | (SEQ ID NO: 2) |
| Oligo 50 5′- ggt agt act tca tgc cgt tct tga g | (SEQ ID NO: 3) |
| Oligo 51 5′- ggg tgt ctg tgt ctc ggt aga cct ggc tat ctg tgt t | (SEQ ID NO: 4) |
| Oligo 52 5′- cta ccg ggc atc tga gta tcg gTa g | (SEQ ID NO: 5) |
| Oligo 53 5′- cga ctg ggc atc tga gta tca gTc g | (SEQ ID NO: 6) |
| Oligo 54 5′- gta ccg gag gac tgt gtt tcg gTa c | (SEQ ID NO: 7) |
| Oligo 55 5′- caa ccg gag gac tgt gtt tcg gTt g | (SEQ ID NO: 8) |
| Oligo 56 5′- caa ccg gag gac tgt gtt tcg gTt g | (SEQ ID NO: 9) |
| Oligo 57 5′- gac cgg agg act gtg ttt cgg Tc | (SEQ ID NO: 10) |
| Oligo 58 5′- cac cgg agg act gtg ttt cgg Tg | (SEQ ID NO: 11) |

### EXAMPLE 4

### Allele Specific Amplification ±SsO7d

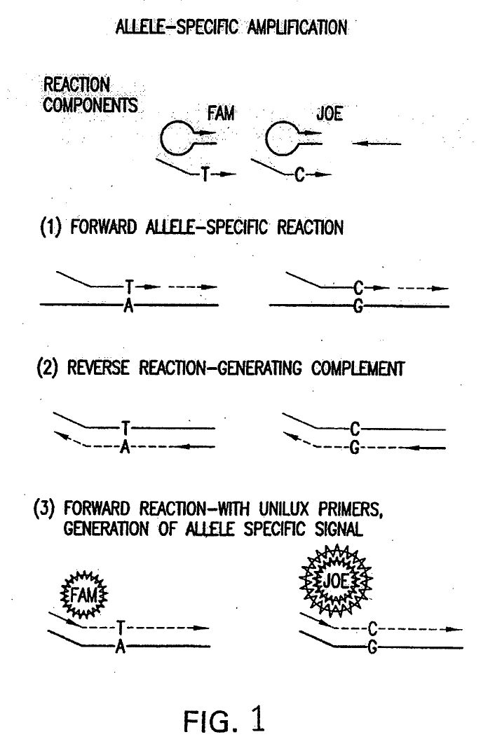

The single nucleotide polymorphism (SNP) genotyping system, one embodiment of which is called the “LUX™ SNP genotyping system” (Light Upon Extension), is described in Example 3. This system combines the use of universal fluorescent detection primers (universal LUX™ primers) with allele specific amplification (ASA) to produce a simple fluorescent assay for the characterization of SNPs. Assays are designed in a multiplex format that detect both alleles in one reaction using two universal LUX™ primers, each labeled with a different fluorophore, and three unlabeled primers. Two allele specific primers and a common reverse (or forward) primer are designed to amplify the polymorphism. The two allele specific primers each have a 5′ extension identical to one of the universal LUX™ primers and the 3′ terminal nucleotide is specific to the mutant allele. Both primers match one allelic variant but are mismatched to the other. The mismatch at the 3′ end of the primer results in the preferential amplification of the matched allele. As the PCR reaction proceeds, the universal LUX™ primers hybridize to the complement of the 5′ extensions to generate a fluorescence signal. Genotyping is based on whether there is a signal in one or both reactions. If only one allele is present, only one signal is produced corresponding to the fluorophore with which one universal primer is labeled. A heterozygote will result in both signals corresponding to both fluorophores. The system allows for the characterization of any polymorphism within a DNA sequence since detection of each SNP only involves the synthesis of three unmodified oligonucleotides. The SNP genotyping system is schematically represented in FIG. 1 using the fluorophores FAM and JOE. One of ordinary skill in the art will realize that the use of FAM and JOE is only representative, and that any fluorophore may be used in the genotyping system described in this example, including those listed in Table 1.

Two universal primers were designed with different fluorophores, FAM or JOE. Although FAM and JOE are exemplified herein, any two fluorophores may be used that can be discriminated by fluorescence detection methods. Both primers were designed to have roughly the same length, G+C content and melting temperature. Primers were “blasted” against public databases to confirm that they did not recognize any sequences within the human genome. The primers are shown in Table 4.

TABLE 4

| | | | | |
|---|---|---|---|---|
| Primer | Sequence | bp | G + C % | Tm |

| | | | | |
|---|---|---|---|---|
| Universal FAM | caagaaATGAAGCGACCATAGGTAACTTCT[FAM]G | 32 | 40.6 | 59.1 |
| | (SEQ ID NO: 12) | | | |
| Universal JOE | gaacgacagctcaatagagataacgtcgt[JOE]C | 31 | 45.2 | 59.9 |
| | (SEQ ID NO: 13) | | | |

Lower case letters indicate the hairpin region
FAM and JOE fluorophores lie on a T nucleotide

Unlabeled primers with the 5′ universal extensions were designed to have the polymorphism on the terminal 3′-nucleotide. The primers were designed by taking the 20 nucleotides directly preceding the SNP site and adding the appropriate 5′ extension:

FAM extension:

(SEQ ID NO: 14)

ATGAAGCGACCATAGGTAACTTCTTG-allele specific
sequence 1
JOE extension:

(SEQ ID NO: 15)

CAGCTCAATAGAGATAACGTCGTTC-allele specific sequence
2

The FAM extension (SEQ ID NO: 14) has allele specific sequence 1. The JOE extension (SEQ ID NO: 15) has allele specific sequence 2. The common primer was designed using Primer3 primer design software from the Whitehead Institute. Primers and unlabeled counterparts were synthesized by CPI (Frederick, Md.). The common reverse primer and universal primers were used in 10-fold excess over the allele specific extended primers.

Panels of genomic DNA containing wild-type, mutant and heterozygous genotypes were obtained from the Coriell Institute (Panel #M08PDR, Coriell Institute, Camden, N.J.). Each sample contained 25 μg of DNA per vial and had an OD260/280 ratio of 1.8±0.1. Genomic DNA (5 ng) was used in each PCR reaction. Synthetic templates were also used for genes when genomic DNA samples were not available. Templates were produced using overlap PCR. Two templates for each gene were produced, one containing the wild-type allele and the other the mutant allele. The two templates were mixed in equal proportions to produce the heterozygotes.

SsO7d was purified from Sulfolobus solfataricus as described previously for SSO P2 RNase (Fusi, et al, Gene, 154:99-103, 1995). The purification was performed by a single step procedure consisting of DEAE-Sephacel chromatography performed at pH 9.3. SsO7d was eluted in the void volume while all the other proteins were bound to the resin. The purification procedure yielded homogeneous SsO7d as determined by SDS-PAGE. The concentration of SsO7d was 0.35 mg/ml. SsO7d was titrated in Super-Mix UDG (#11730-017, Invitrogen Corporation) from 10 ng/μl to 10 pg/μl.

Real-time PCR was performed on an ABI Prism® 7900 instrument (Applied Biosystems) in 20 μl reactions using the universal LUXTM SNP genotyping system. Assays were performed with 2X Platinum Quantitative PCR Super-Mix UDG (#11730-017, Invitrogen) or 2X SNP-Super-mix (60 U/ml Platinum Taq DNA polymerase, Invitrogen #10966-034; 40 mM Tris-HCl., pH 8.4; 100 mM KCl; 6 mM MgCl2; 400 PM dGTP, dATP and dCTP; 800 μM dUTP, #D-0184, Sigma-Aldrich; 40 U/ml uracil-DNA-glycosylase (Invitrogen #18054-015); 0.08 ng/μl SsO7d; 0.2% poly-2-ethyl-2 oxazoline5000[aquazol 5 or J1], Polymer Chemistry Innovations). The following assay composition was used:

Universal LUX primer assays: 10 μl 2X mix, 0.04 μl allele specific primer 1, 0.04 μl allele specific primer 2, 0.4 μl common primer, 0.4 μl universal FAM primer, 0.4 μl universal JOE primer, 0.4 μl ROX reference dye (#12223-012, Invitrogen), DEPC water to 20 μl).

Assays were run with 5 ng of the appropriate genomic DNA sample or 1,000 copies of each synthetic template. PCR reactions using Super-Mix UDG or SNP Super-mix were incubated at 50° C. for 2 minutes, at 95° C. for 2 minutes, then cycled for 40-50 cycles at 92° C. for 15 seconds and 65° C. for 30 seconds. Melting curve analysis was done on LUX™ reactions by incubating at 45° C. for 1 min and then ramping to 95° C. over a 2% ramp rate, followed by incubation at 25° C. for 2 min. Fluorescence was monitored at every PCR cycle and the ramp from 45° C. to 95° C. of the melting curve. At the completion of each PCR run, an end-point plate read was done on the ABI 7900 at 60° C. for 2 minutes. Fluorescent data was graphed on a scatter plot using SDS 2.1 software

Genotyping was performed on three human genes, HLA-b associated transcript 1 (BAT1), HLA-b associated transcript 3 (BAT3) and CCNB 1 (P7). The primers used for these three genes are shown in Table 5. In the table, the first two primers for each gene are the allele-specific primers, and the third is the common (reverse) primer.

TABLE 5

| | | |
|---|---|---|
| Primer | Sequence | SEQ ID NO: |

| | | |
|---|---|---|
| BAT1-G | Caaccggaagtgaaggcag | 16 |
| BAT1-C | Caaccggaagtgaaggcac | 17 |
| BAT1-rev | Taaacagggagagcgcgtat | 18 |
| BAT3-A | Cactggggggaaacttaggca | 19 |
| BAT3-G | Cactggggggaaacttaggcg | 20 |
| BAT3-rev | Acagatgaggaggcaccaag | 21 |
| CCNB1-T | Cggctgttggtttctgctggg | 22 |
| CCNB1-G | Cggctgttggtttctgctggg | 23 |
| CCNB1-rev | Ccatggcttcctcttcacc | 24 |

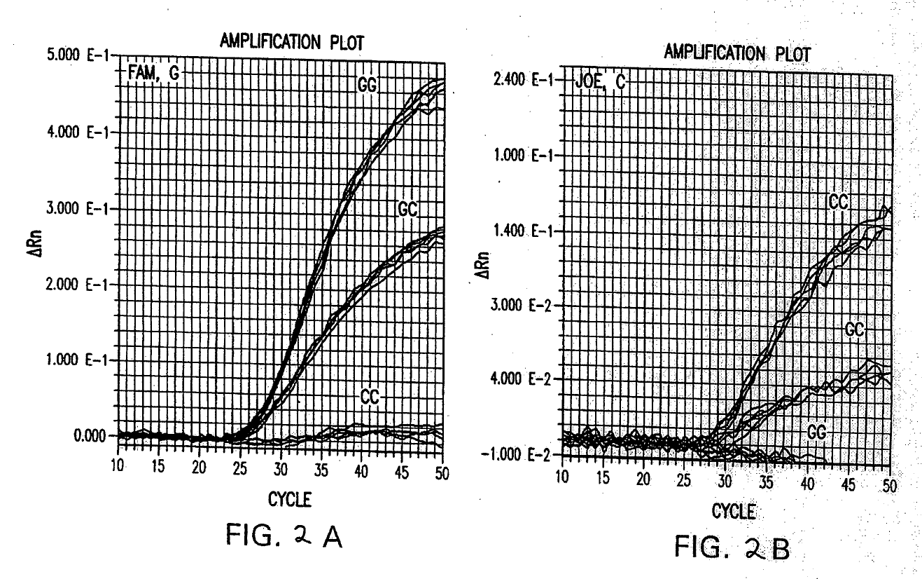

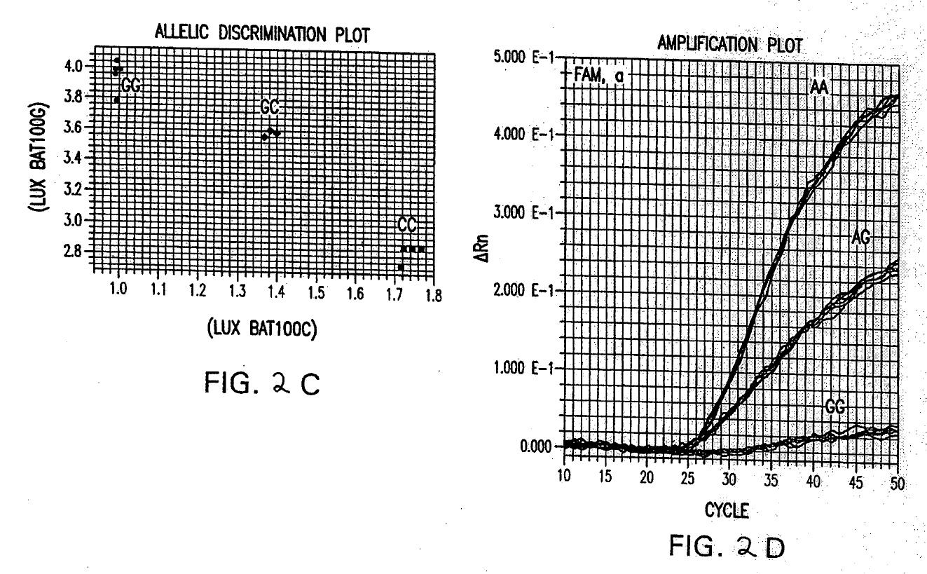

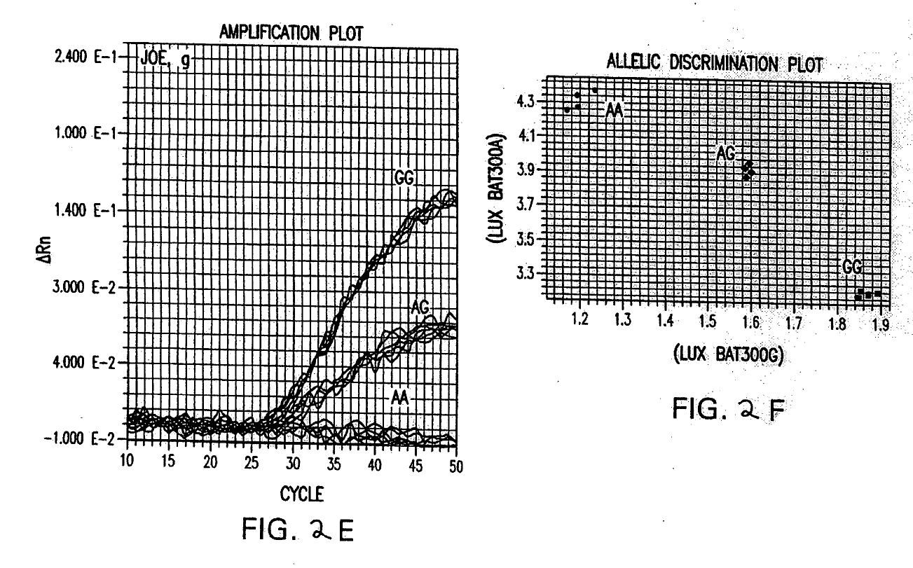

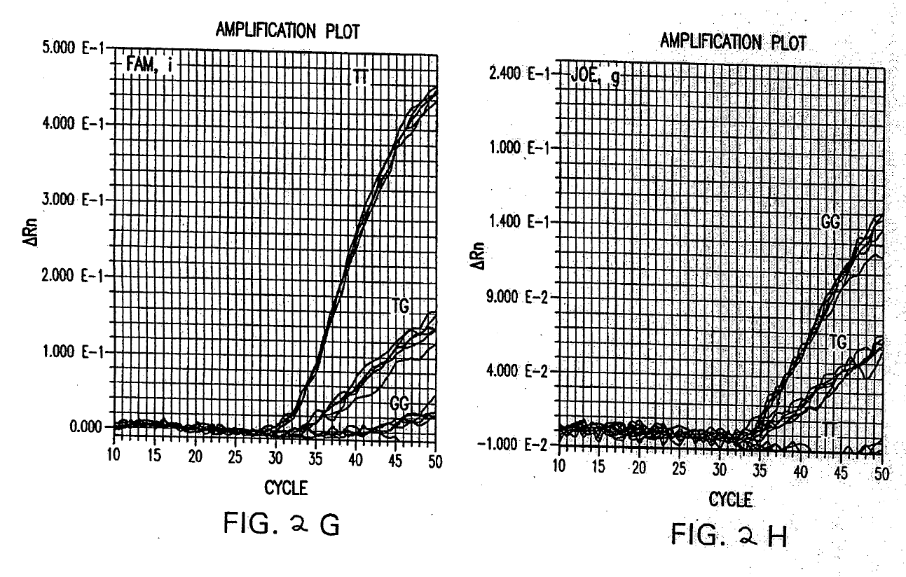

Representative fluorescence plots showing genotyping results using this Universal LUX™ system are shown in FIGS. 2A-F. Real-time amplification plots show specificity in each assay. FIGS. 2A-B shows the results of analysis of a G/C polymorphism in the BAT1 gene. In FIGS. 2A-B, the universal FAM primer is designed to only amplify the G allele and the universal JOE primer is designed to only amplify the C allele. A significant FAM signal is only generated from the GG homozygote and no signal is generated from the GG homozygote. An intermediate signal is seen in both FAM and JOE for the GC heterozygote, since both primer sets are able to amplify one of the two alleles. FIGS. 2C-D show analysis of an A/G polymorphism in the human BAT3 gene. FIGS. 2E-F show analysis of a T/G polymorphism in the human CCNB1 (P7) gene.

The allelic discrimination plots corresponding to FIGS. 2A-F are shown in FIGS. 2G-I FIG. 2G corresponds to FIGS. 2A-B; FIG. 2H corresponds to FIGS. 28C-D; and FIG. 2I corresponds to FIGS. 2E-F. On each axis is the relative fluorescence reading of each dye, FAM on the Y-axis and JOE on the X-axis. Each point represents a DNA sample and is plotted on the graph according to the intensity of each fluorescent signal present after PCR. Samples with strong FAM signals will appear higher on the Y-axis and samples with strong JOE signals will appear further right on the X-axis. Each assay produces three distinct clusters within the plot corresponding to the potential genotypes present. Genotypes are called based on the position of each sample within the plot. The homozygotes are distributed along the X- and Y-axis and the heterozygotes are positioned between the two homozygotes. In FIG. 2G, the GG homozygote produces a strong FAM signal with no JOE signal and is plotted in the upper left. The CC homozygote produces a strong JOE signal with no FAM signal and is plotted in the lower right. The GC heterozygote produces signals of medium intensity in both FAM and JOE and is plotted in the center, approximately on the bisector of the other two clusters. FIGS. 2H and 2I show similar patterns. In these allelic discrimination plots, as more samples are screened the size of each cluster will increase. If the clusters begin to overlap, discriminating between genotypes becomes more difficult. Increasing the relative distance between clusters makes overlap less likely and facilitates genotyping.

In normal conditions, the universal hairpin primers alternate between the hairpin configuration and the single stranded unhybridized conformation. Dissociation of the hairpin results in a slight dequenching of the fluorophore and an increase in the background fluorescence.

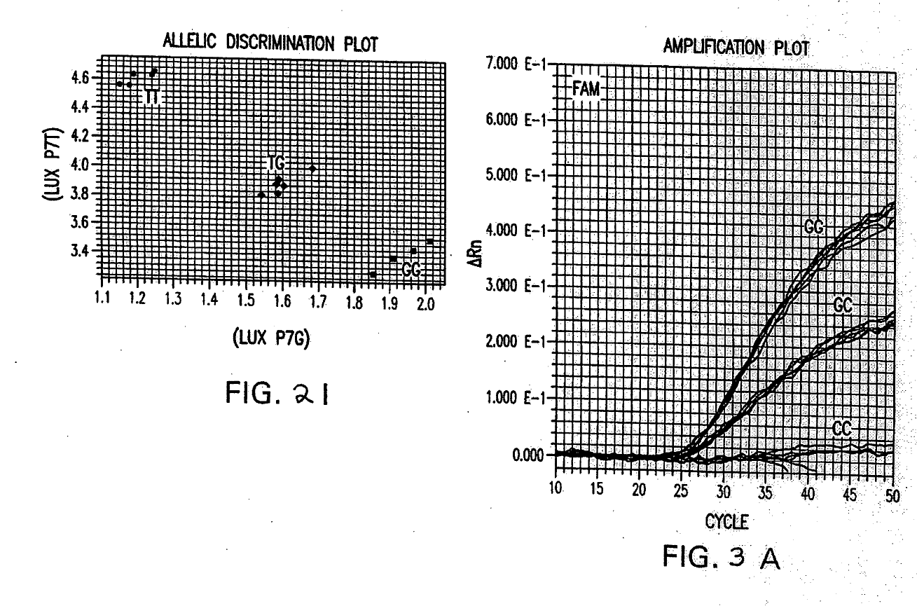

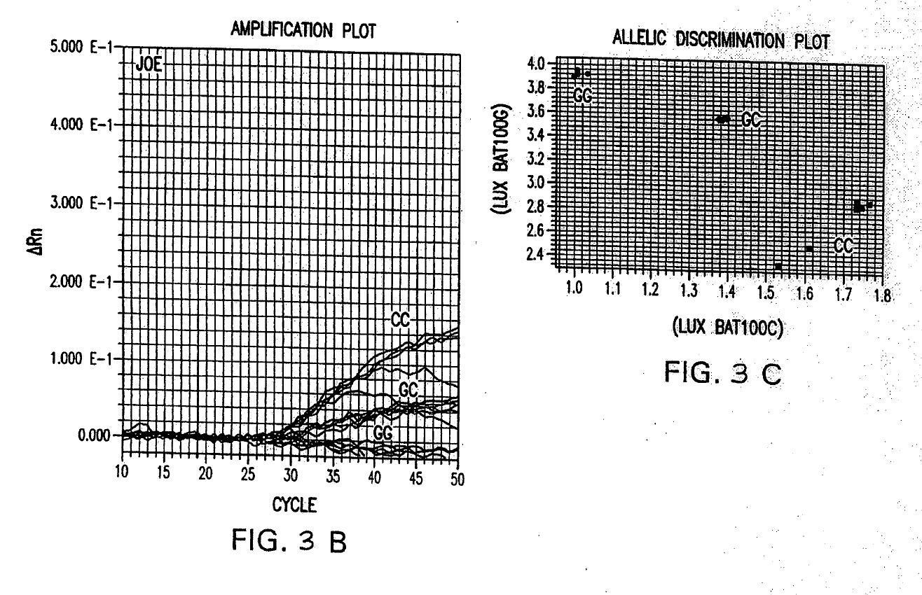

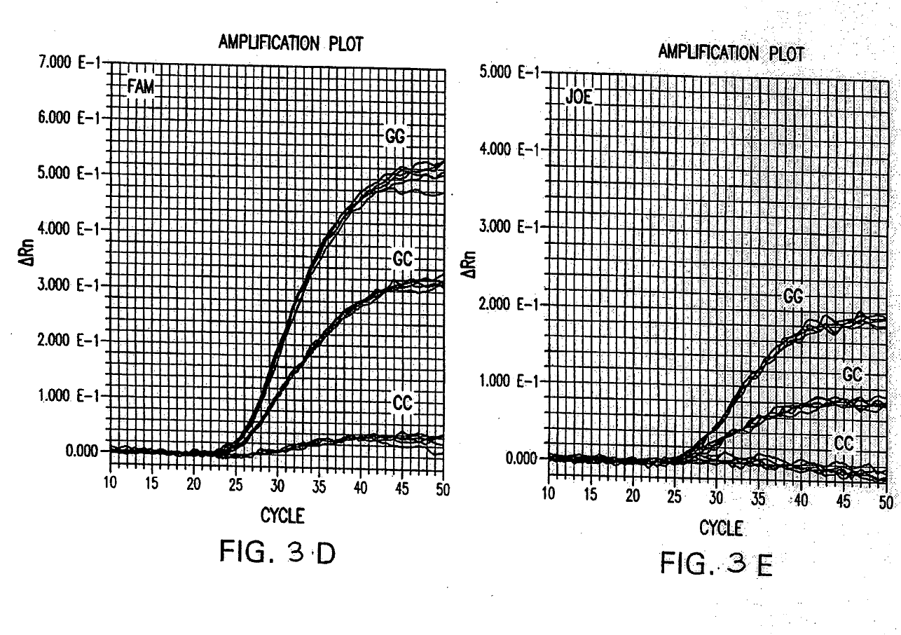

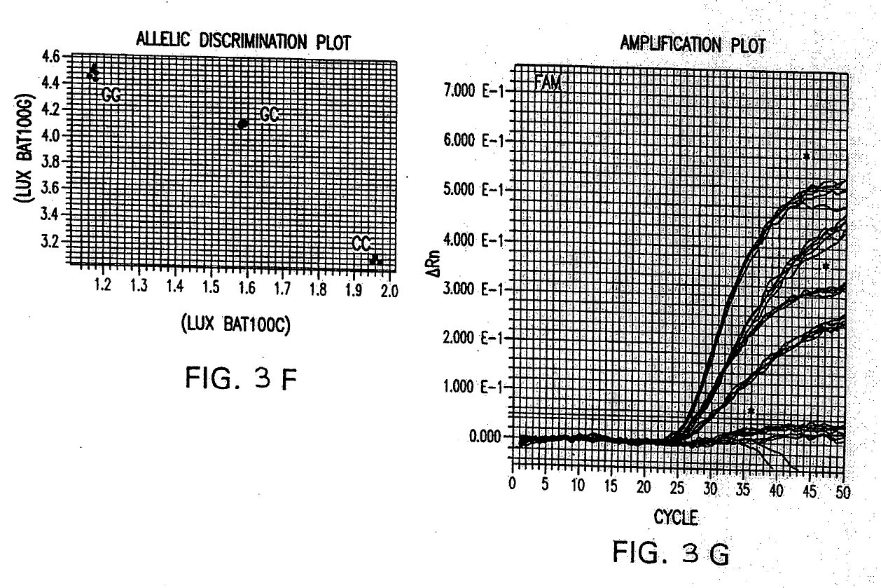

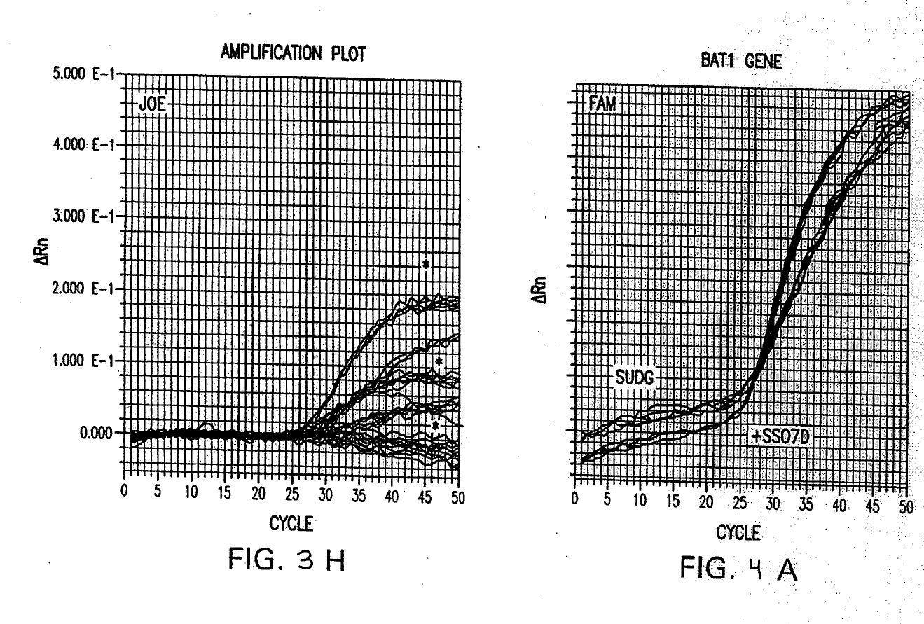

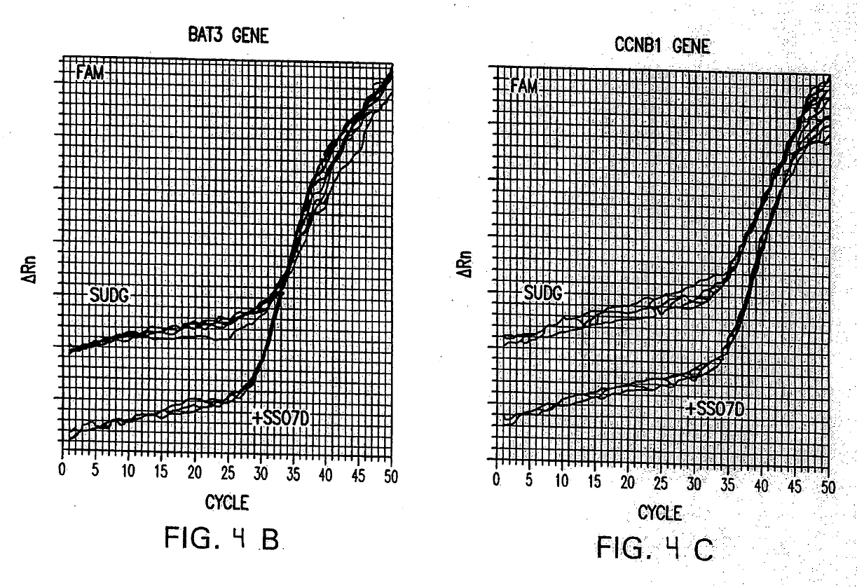

An enzyme mix comprising SsO7d (SNP Super-mix) was used for SNP genotyping of the human BAT1 gene, and compared to SNP genotyping using Platinum Quantitative SuperMix-UDG (#117300-017, Invitrogen) which lacks SsO7d. Use of SsO7d resulted in improved fluorescent signals in real-time and decreased scattering of replicates at end-point. FIG. 3 shows a representative example of results obtained using the SNP Super-mix compared to Platinum Quantitative SuperMix-UDG. FIGS. 3A-B show real-time fluorescence plots, and FIG. 3C shows the allelic discrimination plot, using Platinum Quantitative SuperMix-UDG (no SsO7d). FIGS. 3D-E show real-time fluorescence plots, and FIG. 3F shows the allelic discrimination plot, using SNP Super-mix (comprising SsO7d). A comparison of Super-mix UDG and SNP Super-mix is shown in FIGS. 3G-H. The asterisks in FIGS. 3G-H denote SNP Super-mix. FIGS. 4A-C show raw fluorescence data for the FAM channel for the three SNPs shown previously. Each graph shows the reduction of the background fluorescence with the addition of SsO7d. The reduction in background results in a greater increase of fluorescent signal, improving discrimination of alleles.

The addition of SsO7d to PCR reactions using the universal primers resulted in a greater increase in fluorescence. SsO7d is also highly thermostable and can survive multiple PCR cycles wherein each cycle it can bind to all non-annealed universal primers to maintain the hairpin. The increase in fluorescence results in better discrimination of the two alleles in SNP genotyping assays, as well as in other PCR based SNP genotyping systems such as the 5′ fluorogenic nuclease assay.

In the improved real-time PCR (qPCR) reactions described below, a sample containing a nucleic acid sequence that is to be amplified is mixed with primers that are complementary to sequences that flank the sequence to be amplified, a thermostable polymerase (e.g., Taq, Pfu, Pfx), dNTPs, and a nonspecific double-stranded DNA binding protein (e.g., SsO7d). The normal steps of PCR are then followed: melting, annealing and synthesis by thermal cycling of the mixture. Temperatures, times and numbers of cycles will vary depending on the particular reaction being performed and can be optimized by one of ordinary skill in the art.

In another embodiment, a kit is provided which comprises a thermostable polymerase, dNTPs, PCR reaction buffer and a nonspecific double-stranded DNA binding protein (e.g., SsO7d). The components may be provided in separate containers, or various combinations of components may be provided in the same container.

The following examples illustrate the enhancement of PCR efficiency, specificity and sensitivity using the nonspecific double-stranded DNA binding protein SsO7d.

### EXAMPLE 5

### Enhancement of PCR with SsO7d

Primers specific for the housekeeping gene hydroxymethylbilane synthase (HMBS) were used with the plasmid template at 1×10⁷, 1×10⁶, 1×10⁵, 1×10⁴, 1×10³, 1×10² and 1×10¹ copies per reaction. The plasmid template was qPCR Plasmid Standards Invitrogen Catalog No. 11741-100). The primers used were:

(SEQ ID NO: 25)

HMBS-F: 5′-TGAGAGTGATTCGCGTGGGTAC-3′;
and

(SEQ ID NO: 26)

HMBS-R: 5′-GCTTTCAATGTTGCCACCACA-3′.

qPCR was done in the presence of Taq polymerase (Invitrogen) and SYBR Green I (Invitrogen) with real-time detection on the Roche LightCycler. PCR was performed in 25 μl of PLATINUM® Taq Reaction Buffer with 0.5 U of PLATINUM® Taq, 0.2 mM dNTPs, 0.2 VM forward and reverse primers, and 1.75 mM MgCl₂ using 10⁴-10⁶ copies of target. PLATINUM® Tsp was used under the same conditions. Thermal cycling was performed on 9600 or ABI PRISM® 7700 Sequence Detector (Perkin Elmer). Unless otherwise indicated, in all examples, amplification was conducted with PLATINUM® Taq (available from Invitrogen Corporation (Life Technologies Division)).

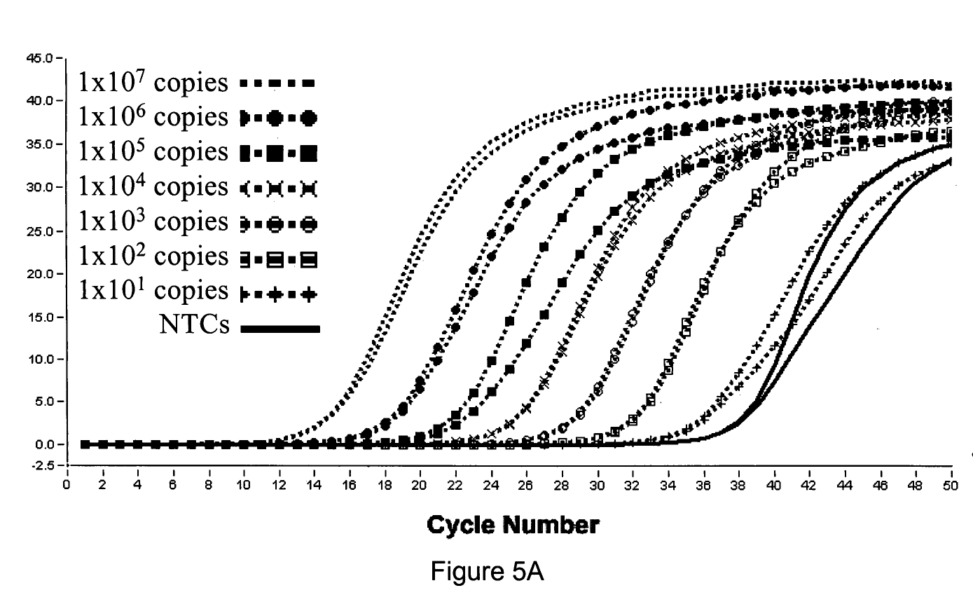

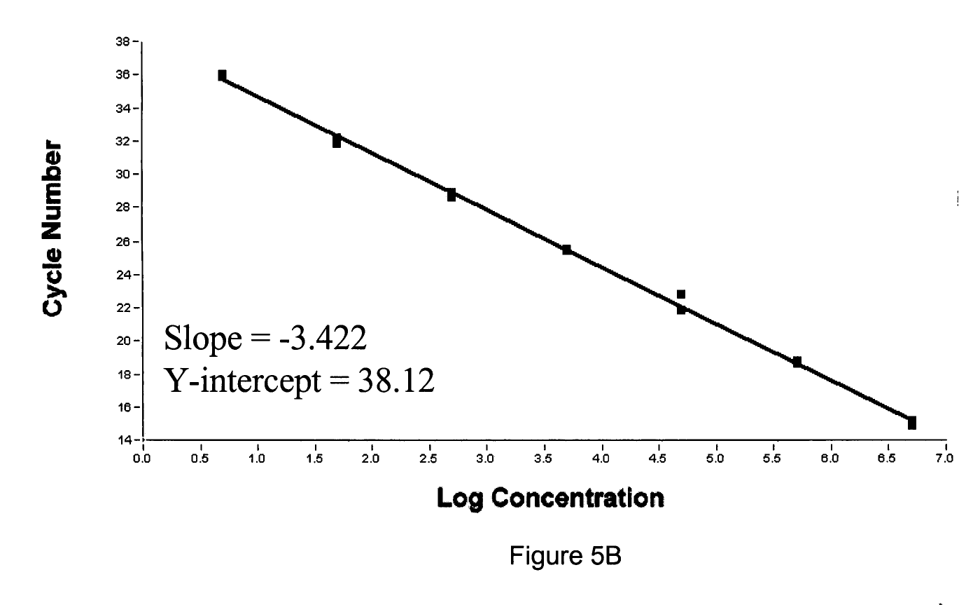

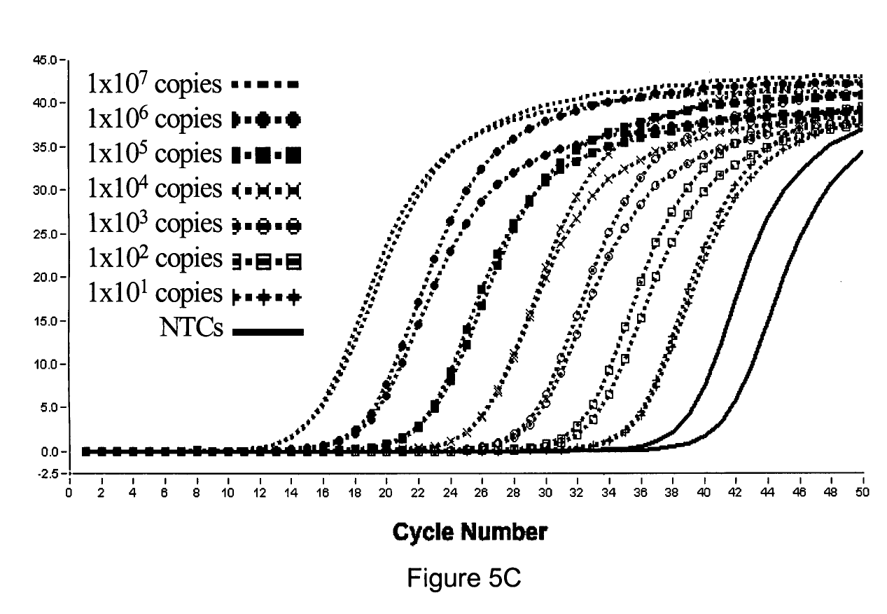

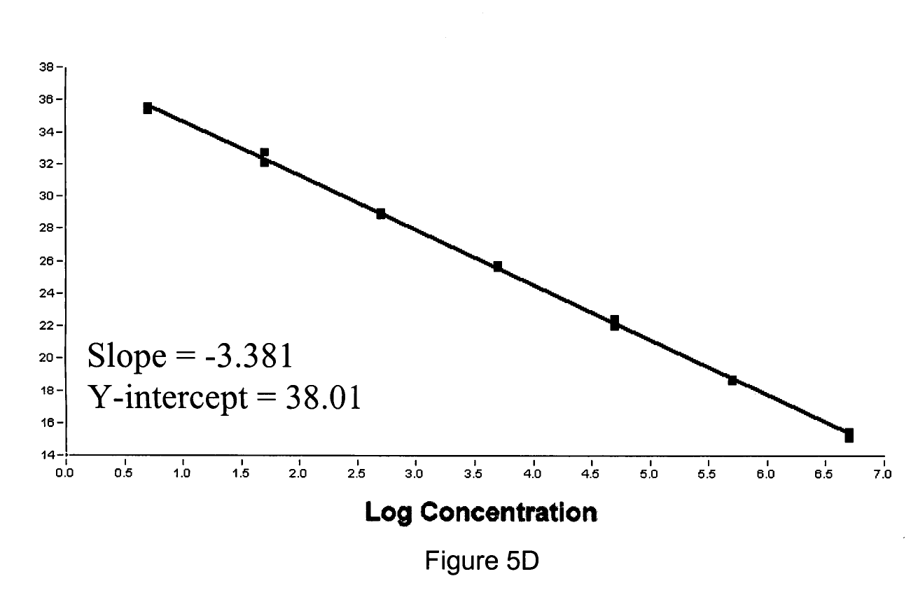

The cycling conditions were: hold at 50° C. for 2 minutes; hold at 92° C. for 2 minutes; 45 cycles of 92° C. for 5 seconds, 55° C. for 20 seconds (single acquisition), 72° C.for 10 seconds; hold at 92° C. for 0 seconds; hold at 55° C. for 10 seconds going to 95° C. for 10 seconds. The slope was set at 0.1° C./second. FIGS. 5A and 5C show amplification plots in the presence (FIG. 5A) and absence (FIG. 5C) of SsO7d. NTC (no template control) products are shown and are a measure of PCR specificity, since the later these appear, the more specific the PCR reaction. NTCs were detected at a later cycle threshold (Ct) in the presence of SsO7d, from 37.98±0.01 without SsO7d (FIG. 5C) to 39.76±1.66 with SsO7d (FIG. 5A). The Ct is the number of cycles required for each sample to reach a detectable signal level.

Standard curves were generated based on the amplification curves in FIG. 5A and FIG. 5C, which are shown in FIG. 5B and FIG. 5D, respectively. The closer the slopes of the standard curves are to −3.32, the more efficient the reaction. The addition of SsO7d resulted in an increase in PCR efficiency from 96.0% (−3.422) without SsO7d to 97.6% (−3.381) with SsO7d.

### EXAMPLE 6

### Dose-Dependent Effect of SsO7d on PCR Specificity

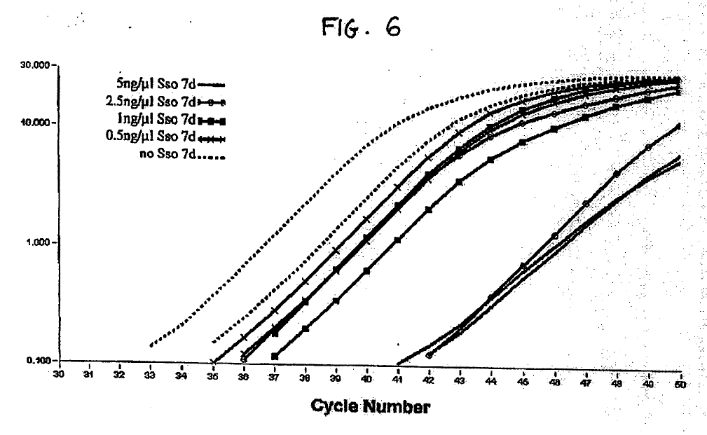

Formation of nonspecific PCR reaction products called primer dimers is often a problem in PCR reactions. In order to determine the effect of SsO7d on primer dimmer formation, HMBS primers with no template added were subjected to 50 PCR cycles with Taq and SYBR Green I detection in real-time as described above. As shown in FIG. 6, SsO7d suppressed primer dimmer formation in a dose-dependent manner, thus increasing the specificity of the PCR reaction. The Ct values are shown in Table 6. The Ct average, representing the later appearance time of primer dimers, increased throughout the SsO7d dose range.

TABLE 6

| SsO7d concentration (ng/μl) | Ct average |
|---|---|
| 0 | 37.57 ± 1.31 |
| 0.5 | 39.47 ± 0.47 |
| 1.0 | 40.22 ± 0.73 |
| 2.5 | 42.61 ± 4.12 |
| 5.0 | 45.93 ± 0.11 |

### EXAMPLE 7

### Enhancement of Pfx Polymerase PCR with SsO7d

Pfx polymerase, which has a 3′-5′ exonuclease activity, was added to a PCR reaction, with real-time detection using SYBR Green I as described above. Pjx, with its 3′ to 5′ exonuclease activity, is able to digest primers and thus inhibit PCR amplification. The PCR reaction was performed using primers specific for the housekeeping gene ribosomal protein L4 (RPL4) and plasmid template (qPCR Plasmid Standards, High Abundance, Invitrogen Catalog #11740-100) at 1×10⁷ copies per reaction. Primers used were:

(SEQ ID NO: 27)

RPL4-F: 5′-AAGCCGCTTCCCTCAAGAGT-3′

(SEQ ID NO: 28)

RPL4-R: 5′-TGGATCTTCTTGCGTGGTGCTC-3′

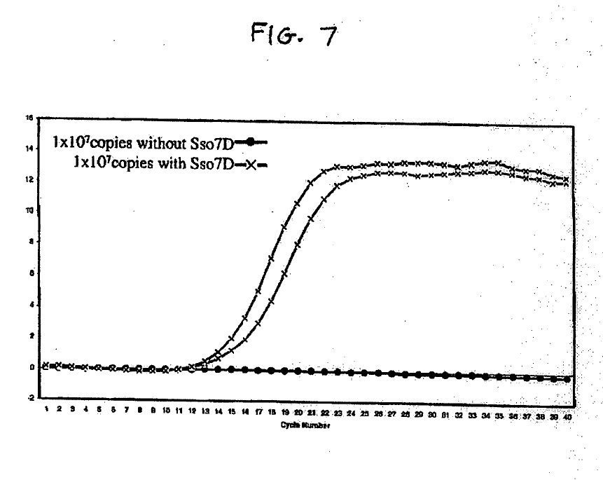

The PCR reaction was performed in Pfx 1× buffer with 1.5 mM MgCl2, 30 U/ml Platinum Taq (Invitrogen) and 3 U/ml of Pfx polymerase (Invitrogen) with or without SsO7d. PCR was done on the ABI 7000 with the following cycling conditions for qPCR: hold at 50° C. for 2 min; hold at 95° C. for 2 min; 40 cycles of 95° C., 15 sec; 60° C., 45 sec. The results are shown in FIG. 7. Duplicate reactions without SsO7d were not amplified, while all four reactions containing SsO7d were amplified. Correct amplification products were confirmed by melting curve analysis and 2% agarose gel electrophoresis.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

What is claimed is:

1. A method for amplification of a DNA molecule, comprising contacting said DNA molecule with a thermostable nucleic acid polymerase and a nonspecific double-stranded DNA binding protein, whereby said nucleic acid molecule is amplified.

2. A composition comprising a nucleic acid molecule, a thermostable nucleic acid polymerase and a nonspecific double-stranded DNA binding protein.


SEQUENCE LISTING (28 sequences)

SEQ ID NO: 1 - DNA, 25 bp, Artificial Sequence
  Synthetic universal primer used to detect any gene-specific nucleotide target sequence.
ctaccgggtgtctgtgtctcggtag

SEQ ID NO: 2 - DNA, 37 bp, Artificial Sequence
  Synthetic universal primer used to detect any gene-specific nucleotide target sequence.
gggtgtctgtgtctcggtagacctggctatctgtgtc

SEQ ID NO: 3 - DNA, 25 bp, Artificial Sequence
  Synthetic universal primer used to detect any gene-specific nucleotide target sequence.
ggtagtacttcatgccgttcttgag

SEQ ID NO: 4 - DNA, 37 bp, Artificial Sequence
  Synthetic universal primer used to detect any gene-specific nucleotide target sequence.
gggtgtctgtgtctcggtagacctggctatctgtgtt

SEQ ID NO: 5 - DNA, 25 bp, Artificial Sequence
  Synthetic universal primer used to detect any gene-specific nucleotide target sequence.
ctaccgggcatctgagtatcggtag

SEQ ID NO: 6 - DNA, 25 bp, Artificial Sequence
  Synthetic universal primer used to detect any gene-specific nucleotide target sequence.
cgactgggcatctgagtatcagtcg

SEQ ID NO: 7 - DNA, 25 bp, Artificial Sequence
  Synthetic universal primer used to detect any gene-specific nucleotide target sequence.
gtaccggaggactgtgtttcggtac

SEQ ID NO: 8 - DNA, 25 bp, Artificial Sequence
  Synthetic universal primer used to detect any gene-specific nucleotide target sequence.
caaccggaggactgtgtttcggttg

SEQ ID NO: 9 - DNA, 25 bp, Artificial Sequence
  Synthetic universal primer used to detect any gene-specific nucleotide target sequence.
caaccggaggactgtgtttcggttg

SEQ ID NO: 10 - DNA, 23 bp, Artificial Sequence
  Synthetic universal primer used to detect any gene-specific nucleotide target sequence.
gaccggaggactgtgtttcggtc

SEQ ID NO: 11 - DNA, 23 bp, Artificial Sequence
  Synthetic universal primer used to detect any gene-specific nucleotide target sequence.
caccggaggactgtgtttcggtg

SEQ ID NO: 12 - DNA, 31 bp, Artificial Sequence
  Synthetic universal primer with fluorophore used to detect any gene-specific nucleotide target sequence.
caagaaatgaagcgaccataggtaacttctg

SEQ ID NO: 13 - DNA, 30 bp, Artificial Sequence
  Synthetic universal primer with fluorophore used to detect any gene-specific nucleotide target sequence.
gaacgacagctcaatagagataacgtcgtc

SEQ ID NO: 14 - DNA, 26 bp, Artificial Sequence
  Fluorophore extension attached to synthetic universal primer, used to detect any gene-specific nucleotide target sequence.
atgaagcgaccataggtaacttcttg

SEQ ID NO: 15 - DNA, 25 bp, Artificial Sequence
  Fluorophore extension attached to synthetic universal primer, used to detect any gene-specific nucleotide target sequence.
cagctcaatagagataacgtcgttc

SEQ ID NO: 16 - DNA, 19 bp, Artificial Sequence
  Synthetic allele-specific primer used to detect any gene-specific nucleotide target sequence.
caaccggaagtgaaggcag

SEQ ID NO: 17 - DNA, 19 bp, Artificial Sequence
  Synthetic allele-specific primer used to detect any gene-specific nucleotide target sequence.
caaccggaagtgaaggcac

SEQ ID NO: 18 - DNA, 20 bp, Artificial Sequence
  Synthetic common (reverse) primer used to detect any gene-specific nucleotide target sequence.
taaacagggagagcgcgtat

SEQ ID NO: 19 - DNA, 21 bp, Artificial Sequence
  Synthetic allele-specific primer used to detect any gene-specific nucleotide target sequence.
cactggggggaaacttaggca

SEQ ID NO: 20 - DNA, 21 bp, Artificial Sequence
  Synthetic allele-specific primer used to detect any gene-specific nucleotide target sequence.
cactggggggaaacttaggcg

SEQ ID NO: 21 - DNA, 20 bp, Artificial Sequence
  Synthetic common (reverse) primer used to detect any gene-specific nucleotide target sequence.
acagatgaggaggcaccaag

SEQ ID NO: 22 - DNA, 21 bp, Artificial Sequence
  Synthetic allele-specific primer used to detect any gene-specific nucleotide target sequence.
cggctgttggtttctgctggg

SEQ ID NO: 23 - DNA, 21 bp, Artificial Sequence
  Synthetic allele-specific primer used to detect any gene-specific nucleotide target sequence.
cggctgttggtttctgctggg

SEQ ID NO: 24 - DNA, 19 bp, Artificial Sequence
  Synthetic common (reverse) primer used to detect any gene-specific nucleotide target sequence.
ccatggcttcctcttcacc

SEQ ID NO: 25 - DNA, 22 bp, Artificial Sequence
  Synthetic primer specific for a housekeeping gene used to detect any gene-specific nucleotide target sequence.
tgagagtgattcgcgtgggtac

SEQ ID NO: 26 - DNA, 21 bp, Artificial Sequence
  Synthetic primer specific for a housekeeping gene used to detect any gene-specific nucleotide target sequence.
gctttcaatgttgccaccaca

SEQ ID NO: 27 - DNA, 20 bp, Artificial Sequence
  Synthetic primer specific for a housekeeping gene and plasmid template used to detect any gene-specific nucleotide target sequence.
aagccgcttccctcaagagt

SEQ ID NO: 28 - DNA, 22 bp, Artificial Sequence
  Synthetic primer specific for a housekeeping gene and plasmid template used to detect any gene-specific nucleotide target sequence.
tggatcttcttgcgtggtgctc